(19) United States
(12) Patent Application Publication
(10) Pub. No.: US 2018/0160985 A1
(43) Pub. Date: Jun. 14, 2018

(54) METHODS AND DEVICES FOR DETERMINING METABOLIC STATES

(71) Applicant: Ultradian Diagnostics LLC, Rensselaer, NY (US)

(72) Inventor: John Patrick Willis, Rensselaer, NY (US)

(73) Assignee: Ultradian Diagnostics LLC, Rensselaer, NY (US)

(21) Appl. No.: 15/576,625

(22) PCT Filed: Jun. 16, 2016
(86) PCT No.: PCT/US2016/037926

§ 371 Date: Nov. 22, 2017

Related U.S. Application Data
(60) Provisional application No. 62/181,762, filed on Jun. 18, 2015.

(51) Int. Cl. A61B 5/00; A61B 5/145; A61B 5/1486; A61B 5/1459; G01N 27/327; G16H 50/20; C12Q 1/00

(52) U.S. Cl. CPC A61B 5/7275; G01N 2800/042; A61B 5/7246; A61B 5/14546; A61B 5/14532; A61B 5/14865; A61B 5/1459; A61B 5/14503; A61B 5/7257; A61B 5/4866; G01N 27/3273; G01N 27/3277; G16H 50/20; C12Q 1/006; A61B 5/4839; A61B 5/7203; A61B 5/7253

(57) ABSTRACT

In vivo or in vitro sensors in combination with an electronic monitor enable the recognition, quantitation, and tracking of metabolic oscillations of living cells immobilized on or in close proximity to an analyte sensor providing methods, systems, and devices to monitor, probe, diagnose or treat abnormal metabolic states. The patterns or fingerprints of metabolic oscillations yield information about analyte concentration and the status of cellular metabolism. The analysis provides early recognition of abnormal metabolic states and provides treatment options that can avoid complications from metabolic disorders such as diabetes.

### CROSS-REFERENCE TO RELATED APPLICATIONS

This application is an international patent application filed in accordance with the patent cooperation treaty. This international application claims priority benefit of U.S. Provisional Patent Application Ser. No. 62/181,762 filed Jun. 18, 2015, and entitled “Systems, Methods, and Devices for Monitoring and Diagnosing Metabolic States.” The disclosure of the aforementioned Provisional Patent Application Ser. No. 62/181,762 is hereby incorporated by reference in its entirety.

### BACKGROUND

In recent years the incidence of diabetes has increased significantly and has been labeled the “silent epidemic”. Since the 1970s, the incidence of obesity has more than doubled to the point where currently, 34% of the US population is obese. This is also reflected in countries that have adopted the eating habits of western societies. Population statistics show that diabetes presently afflicts over 8% of the world population or 390 million people, and 47% are undiagnosed. Of the total, 10% have type 1 and 90% have type 2 diabetes. Diabetes is the leading cause of adult blindness, kidney failure and non-traumatic amputations. People with diabetes are 2-4 times more likely to suffer from heart disease and stroke. The rate of occurrence of diabetes and the rate of diagnosis of diabetes is expected to increase dramatically over the next decades due to an aging population, an increasing rate of obesity, physical inactivity, a rising rate among high-risk minority groups and an increased standard of living in less developed countries.

Metabolic disorders such as diabetes, metabolic syndrome, and various aberrations of glycolytic metabolism are of great concern due to the long term and chronic health effects such disorders cause. Early diagnosis and subsequent treatment can be very helpful in providing better outcomes for persons with respect to these conditions whether they are recognized as disease states or pre-disease states.

### SUMMARY

Monitoring and diagnosing glucose metabolism at various levels of heath including normal, impaired glucose tolerance, type 2 diabetes, type 1 diabetes, and metabolic syndrome is an area of intense interest because early diagnosis, intervention and an intensified program for tightly controlling blood glucose levels dramatically reduce complications such as those from poorly regulated diabetes. There is a need for easy to use systems, methods, and devices that enable accurate diagnosing of various stages of diabetes, as well as other metabolic disorders. This disclosure provides a unique and unexpected approach to monitor, probe, and diagnose metabolic states or fingerprints by, in some embodiments, detecting, following, and analyzing patterns of metabolic oscillations.

The methods, systems, and devices disclosed herein enable the recognition of metabolic oscillations that prior to this disclosure have been lost in the background noise in current diagnostic systems, devices, and methods. The lack of recognition of these metabolic oscillations in the currently available processes and procedures represents a loss of an opportunity to use this valuable information for assessing metabolic states of persons. The present disclosure solves this problem by providing methods, systems, and devices that enable the recognition and use of this valuable information for diagnostics, treatment, research, and apparatus or device implementation. Using the information that can be provided by the methods and systems disclosed herein enables the recognition of aberrant metabolic behavior, such as pre-diabetes, under circumstances where aberrant metabolic behavior would not have been detected using the known systems, methods, and devices providing the opportunity to avoid complications from undetected metabolic disorders, better outcomes, and financial savings by the avoidance of expensive chronic care. The methods and systems provided herein by detecting patterns of metabolic oscillations enables the tailoring of medical interventions to individuals because as will be described in detail herein, the fine structures of metabolic processes are often hidden within information relating to the total concentration of a metabolite. In addition to providing a rapid and easily implemented diagnostic test for various metabolic aberrations such as type 1 diabetes, type 2 diabetes, pre-diabetic conditions, and metabolic syndrome that are of significant health concern, the use of the systems, methods, and devices disclosed herein provide for finer analysis of metabolic processes and enable recognition of conditions, including various states of type 1 diabetes, type 2 diabetes, pre-diabetic conditions, and metabolic syndrome, that have not been detectable by current means.

In the case of glucose, with the use of conventional in vivo glucose sensors, cellular glucose oscillations have been lost in the background noise. If these cellular glucose oscillations are not recognized and measured, valuable physiological information that provides opportunities to avoid complications of diabetes is lost. The methods, systems, and devices disclosed herein fill this need by enabling the observation of cellular glucose oscillations in the context of a real time diagnostic of persons, or other subjects. By, in some embodiments, providing a unique insight into the fine structure of metabolism of glucose and other metabolites, this disclosure enables the opportunity to rapidly develop therapeutic regimes tailored to individual persons, or other subjects, in the course of a few hours or days.

Prior to this disclosure, diagnosing metabolic diseases required frequent blood drawing and external analysis of body fluid constituents including metabolites. Because early diagnosis, intervention, and an intensified program for tightly controlling blood glucose levels dramatically reduce complications of diabetes, there is a need for easy to use methods, systems, and devices as taught by this disclosure in the diagnosing of various stages of metabolic disorders such as, in the case of diabetes, assessing persons along the spectrum from normal to impaired glucose tolerance, Metabolic Syndrome, to type 1 or type 2 diabetes. The International Diabetes Foundation defines the metabolic syndrome as a cluster of the most dangerous heart attack risk factors: diabetes and raised fasting plasma glucose, abdominal obesity, high cholesterol and high blood pressure. It is estimated that around 20-25 percent of the world's adult population have the metabolic syndrome and they are twice as likely to die from and three times as likely to have a heart attack or stroke compared with people without the syndrome. In addition, people with metabolic syndrome have a fivefold greater risk of developing type 2 diabetes.

Prior to this disclosure, a variety of laboratory methods were used for diagnosing metabolic diseases. These methods require frequent blood drawing and external analysis of body fluid constituents including metabolites, products of metabolism, electrolytes, enzymes, proteins, DNA, antibodies, antigens and hormones. For example, many of these analyses require a sample such as whole blood, plasma, serum or urine. These types of external body fluid analyses are most often performed when a patient is hospitalized and experiencing symptoms characteristic of a particular disease or diseases. The results of these prior art approaches were inconsistent and overly reliant on maintaining the original state of the sample when it was first drawn. Even with refrigeration or the addition of preservatives, results may not be indicative of cellular processes in vivo. Due to the expense and the need for hospitalization or outpatient medical procedures, these types of analyses are not performed on a routine basis to screen for the early signs of a metabolic disease nor are they performed over a period of time on patients at risk for developing a metabolic disease thus highlighting the need for the approaches described herein.

Another approach for diagnosing diabetes is the oral glucose tolerance test (OGTT) and a variation known as the intravenous glucose tolerance test (IVGTT) for assessing insulin resistance and glucose intolerance. These methods require in-patient testing in a clinic or hospital and frequent blood sampling at 1-3 minute intervals and are time consuming and expensive. In some embodiments, disclosed herein, is a sensor that can be worn on the skin surface to measure glucose within the dermis and be used in accordance with the systems and methods disclosed herein. Using the sensor in a device in accordance with the systems and methods disclosed herein provides in some embodiments for a subject to wear the device for a period of 24 hours or more at home and then either return to a physician's office to have the device removed or the subject can remove the device at home and send it back to the physician in a prepaid mailer, or the subject can wirelessly transmit the data to the physician, wherein cellular metabolic oscillations can be obtained without periodic blood drawing. The data stored within the device are analyzed using proprietary software that can be sold with the device or downloaded and/or provided over the internet.

Several classes of type 2 diabetes have been proposed. For example, there are individuals that exhibit normal fasting glucose (NFG) levels, but postprandial impaired glucose tolerance (IGT). This β-cell abnormality is not revealed by a fasting blood glucose test. There is a need for an easily implemented, routine test for determining those at risk for type 1 and type 2 diabetes early enough for medical intervention to prevent or forestall long-term complications.

In some embodiments, provided herein is a system for measuring cellular metabolic oscillations of a component of cellular metabolism of a subject, comprising a sensor for determining a level of the component of cellular metabolism over a period of time to provide response data, a receiver operably connected to the sensor comprising a computer readable memory configured for receiving and storing the response data, a computer processor operably connected to the receiver comprising executable computer code to obtain a time series comprising amplitude and frequency data from the response data, and means for extracting cellular metabolic oscillations of the component of cellular metabolism of the subject from the time series comprising amplitude and frequency data from the response data. In some embodiments, the component of cellular metabolism is selected from the group consisting of pyruvate, lactate, adenosine triphosphate, adenosine diphosphate, nicotinamide adenine dinucleotide, insulin and combinations thereof. In some embodiments the component of cellular metabolism is glucose. In embodiments where the component of cellular metabolism is glucose, some embodiments as taught by this disclosure enable aberrations of glucose metabolism such as those that relate to various stages and types of diabetes to be assessed and/or diagnosed. Thus, this disclosure, in various embodiments provided herein provides for the recognition of states and stages of glycemic disease states, glycemic normal states, and glycemic pre-disease states. Such glycemic states, in some embodiments, include, for example, various stages of diabetes, type 1 diabetes, type 2 diabetes, pre-diabetic conditions, and metabolic syndrome.

In some embodiments, the system is configured to display changes of the concentration of the component of cellular metabolism over time. In some embodiments, the system further comprises a display and in other embodiments the display is configured to show in graphical form response data, concentration, or metabolic patterns. In some embodiments, a graphical user interface can be used for the display.

In some embodiments, the system further comprises a mounting unit for mounting on a subject's skin. In some embodiments, the sensor comprises an electrochemical cell comprising a working electrode and a counter electrode, a voltage source which provides a voltage between the working electrode and the counter electrode when electrically connected through a conductive medium; and a computing system which measures the dynamic current output from the working electrode.

In some embodiments, the working electrode is coated with a protein layer and a diffusion limiting barrier covering the protein layer. In some embodiments, the voltage source is a potentiostat. In some embodiments, the counter electrode is in contact with a diffusion limiting barrier. In some embodiments, a voltage waveform is applied between the counter electrode and working electrode. In some embodiments, the system further comprises a reference electrode.

In some embodiments, the electrochemical cell comprises an active zone and the active zone comprises the component of cellular metabolism at varying, or at different, concentrations from the bulk concentration of the component of cellular metabolism during potentiostat voltage applications. In some embodiments, the active zone comprises the component of cellular metabolism at concentrations of between about 0% and about 100% of the bulk concentration during potentiostat voltage applications. In some embodiments, the active zone comprises the component of cellular metabolism at concentrations of between about 0% and about 50% of the bulk concentration during potentiostat voltage applications. In some embodiments, the active zone comprises the component of cellular metabolism at concentrations of between about 0% and about 25% of the bulk concentration during potentiostat voltage applications. In some embodiments, the active zone comprises the component of cellular metabolism at concentrations of between about 0% and about 10% of the bulk concentration during potentiostat voltage applications. In some embodiments, the active zone comprises the component of cellular metabolism at concentrations of between about 0% and about 1% of the bulk concentration during potentiostat voltage applications. In some embodiments, the active zone comprises the component of cellular metabolism at concentrations of between about 1% and about 50% of the bulk concentration during potentiostat voltage applications.

In some embodiments, the counter electrode diffusion limiting barrier is the skin of a subject. In some embodiments, the diffusion limiting barrier comprises a polymeric material. In some embodiments the polymeric material comprises a polyurethane. In some embodiments, the protein is glucose oxidase.

In some embodiments, a filter is used as the means for extracting cellular metabolic oscillations from the time series comprising amplitude and frequency data from the response data. In some embodiments, the filter is a wavelet. Various filters can be used for extracting cellular metabolic oscillations from the time series comprising amplitude and frequency data from the response data. In some embodiments, the filter is a moving average filter. In some embodiments, the filter is a low pass filter. In some embodiments, the filter is a recursive filter. In some embodiments obtaining a time series comprises computing a point to point difference in sensor response versus time.

In some embodiments the signal is a voltage. In some embodiments, the signal is a current. In some embodiments the signal is optical. In some embodiments, the signal is electromagnetic energy.

In some embodiments, the sensor is coated with immobilized living cells. In some embodiments, the sensor is in contact with living cells. In some embodiments, the cells are eukaryotic. In some embodiments, the cells are prokaryotic. In some embodiments, the cells are yeast cells. In some embodiments, the cells are mammalian cells. In some embodiments, the cells are cancer cells.

In some embodiments, the sensor is in vitro. In some embodiments, the sensor is in vivo.

In some embodiments, the subject is a mammal. In some embodiments, the subject is a human.

In some embodiments, taken together the sensor for determining a level of the component of cellular metabolism over a period of time to provide response data, the receiver operably connected to the sensor comprising a computer readable memory configured for receiving and storing the response data, and the computer processor operably connected to the receiver comprising executable computer code to obtain a time series comprising amplitude and frequency data from the response data are a continuous glucose monitor or are incorporated in a continuous glucose monitor.

In some embodiments, provided herein is a system for measuring glucose oscillations of a subject comprising a sensor for determining a level of glucose over a period of time to provide response data, a receiver operably connected to the sensor comprising a computer readable memory configured for receiving and storing the response data, a computer processor operably connected to the receiver comprising executable computer code to obtain a time series comprising amplitude and frequency data from the response data, and a means for extracting glucose oscillations from the time series comprising amplitude and frequency data from the response data. In some embodiments, the level of glucose is a concentration.

In some embodiments, the system is configured to display changes of the concentration of glucose over time. In some embodiments, the system further comprising a display and in other embodiments the display is configured to show in graphical form raw response data, concentration, or metabolic patterns. In some embodiments the system can further comprise a mounting unit for mounting on a skin of the subject. In some embodiments, the sensor can comprise an electrochemical cell comprising a working electrode and a counter electrode, a voltage source which provides a voltage between the working electrode and the counter electrode when electrically connected through a conductive medium, and a computing system which measures the dynamic current output from the working electrode. In some embodiments, the working electrode is coated with a protein layer and a diffusion limiting barrier covering the protein layer. In some embodiments, the voltage source is a potentiostat. The potentiostat, in some embodiments, is configured to provide voltage applications. In some embodiments, the potentiostat provides voltage applications. In some embodiments, the counter electrode is in contact with a diffusion limiting barrier. In some embodiments, a voltage waveform is applied between the counter electrode and working electrode. In some embodiments, the system further comprises a reference electrode.

In some embodiments, the system for measuring glucose oscillations of a subject comprises an electrochemical cell which comprises an active zone and the active zone comprises glucose at varying, or at different, concentrations from the bulk concentration of glucose during potentiostat voltage applications. In some embodiments, the active zone comprises glucose at concentrations of between about 0% and about 100% of the bulk concentration of glucose during potentiostat voltage applications. In some embodiments, the active zone comprises glucose at concentrations of between about 0% and about 50% of the bulk concentration of glucose during potentiostat voltage applications. In some embodiments, the active zone comprises glucose at concentrations of between about 0% and about 25% of the bulk concentration of glucose during potentiostat voltage applications. In some embodiments, the active zone comprises glucose at concentrations of between about 0% and about 10% of the bulk concentration of glucose during potentiostat voltage applications. In some embodiments, the active zone comprises glucose at concentrations of between about 0% and about 1% of the bulk concentration of glucose during potentiostat voltage applications. In some embodiments, the active zone comprises glucose at concentrations of between about 1% and about 50% of the bulk concentration of glucose during potentiostat voltage applications. In some embodiments, the counter electrode diffusion limiting barrier is the skin of the subject, in other embodiments, the diffusion limiting barrier comprises a polymeric material, and in some embodiments the polymeric material comprises a polyurethane. In some embodiment, the working electrode is coated with a protein layer and in some embodiments the protein is glucose oxidase.

In some embodiments, the means for extracting cellular glucose oscillations from the time series comprising amplitude and frequency data from the response data is a filter. In some embodiments, the filter is a wavelet, in some embodiments the filter is a moving average filter, in some embodiments, the filter is a low pass filter, in some embodiments, the filter is a recursive filter. In some embodiments, the response data is an analog signal. In some embodiments, obtaining a time series comprises computing a point to point difference in sensor response versus time. In some embodiments, the signal is a voltage, in some embodiments the signal is a current, in some embodiments the signal is optical, and in some embodiment the signal is electromagnetic energy. In some embodiment, the sensor is coated with immobilized living cells selected from eukaryotic cells, prokaryotic cells, yeast cells, mammalian cells or combinations thereof. In some embodiments, the sensor is in vitro while in other embodiments the sensor is in vivo.

In various embodiments disclosed herein, and throughout this application, a potentiostat provides a steady state voltage, voltage pulses, or pulsed voltage, between the working electrode and the counter electrode of the electrochemical cell. In various embodiments the pulsed voltage has a pulse period and a pulse width. In some embodiments, the pulsed voltage has a pulse period of between about 1 second and about 100 minutes. In some embodiments, the pulse period is between about 5 seconds and about 2 minutes. In some embodiments, the pulse period is between about 5 seconds and about 20 seconds. In some embodiments, the pulse width is between about 10 microseconds and about 100 seconds.

In some embodiments, taken together, the sensor for determining a level of glucose over a period of time to provide response data, the receiver operably connected to the sensor comprising a computer readable memory configured for receiving and storing the response data, and the computer processor operably connected to the receiver comprising executable computer code to obtain a time series comprising amplitude and frequency data from the response data are a continuous glucose monitor or are incorporated in a continuous glucose monitor (CGM). That is, in some embodiments, taken together, the sensor, the receiver, and the computer processor are a continuous glucose monitor (CGM) or are incorporated in a continuous glucose monitor (CGM). In some embodiments, provided herein is a continuous glucose monitor (CGM) comprising a sensor for glucose, a receiver operably connected to the sensor comprising a computer readable memory configured for receiving and storing the response data, and the computer processor operably connected to the receiver comprising executable computer code to obtain a time series comprising amplitude and frequency data from the response data.

In some embodiments, provided herein is a system for measuring cellular glucose oscillations of a subject, the system comprising a continuous glucose monitor (CGM) which comprises a receiver operably connected to the continuous glucose monitor comprising a computer readable memory configured for receiving and storing the response data, a computer processor operably connected to the receiver comprising executable computer code to obtain a time series comprising amplitude and frequency data from the response data, and means for extracting cellular metabolic oscillations of the component of cellular metabolism of the subject from the time series comprising amplitude and frequency data from the response data. In some embodiments, the system for measuring cellular glucose oscillations of a subject further comprises a potentiostat providing voltage pulses and the continuous glucose monitor comprises an electrochemical cell which comprises an active zone and the active zone comprises glucose at varying, or at different, concentrations from the bulk concentration of glucose during potentiostat voltage pulses. In some embodiments, the active zone comprises glucose at concentrations of between about 0% and about 100% of the bulk concentration of glucose during potentiostat voltage pulses. In some embodiments, the active zone comprises glucose at concentrations of between about 0% and about 50% of the bulk concentration of glucose during potentiostat voltage pulses. In some embodiments, the active zone comprises glucose at concentrations of between about 0% and about 25% of the bulk concentration of glucose during potentiostat voltage pulses. In some embodiments, the active zone comprises glucose at concentrations of between about 0% and about 10% of the bulk concentration of glucose during potentiostat voltage pulses. In some embodiments, the active zone comprises glucose at concentrations of between about 0% and about 1% of the bulk concentration of glucose during potentiostat voltage pulses. In some embodiments, the active zone comprises glucose at concentrations of between about 1% and about 50% of the bulk concentration of glucose during potentiostat voltage pulses.

In some embodiments, provided herein is a glucose sensor system for monitoring changes in cellular glucose metabolism comprising a continuous glucose sensor configured to produce sensor response data indicative of changes in concentration of glucose at a cell surface of a subject, a receiver configured to receive continuous glucose sensor data, wherein the receiver comprises a computer readable memory for receiving and storing continuous glucose sensor response data; a computer processor operably connected to the receiver configured to process the glucose sensor data to produce processed glucose sensor data, compute a time series comprising glucose amplitude and frequency data from the processed sensor response data, and use the obtained amplitude and frequency data to calibrate the sensor in units of glucose concentration, and a display for visualizing the continuous glucose sensor response data. In some embodiments, the sensor provides response data in the form of an analog signal convertible to the concentration of glucose. In some embodiments, the analog signal is electrical, optical, electromagnetic, or combinations thereof.

In some embodiments, provided is a system for measuring real time changes of glucose concentration of a mammal at a sensor-cellular interface comprising an electrochemical biosensor having an active zone and a diffusion limiting barrier, the electrochemical biosensor configured such that when engaged with subcutaneous tissue of a mammal, glucose concentration at the sensor-cellular interface oscillates between about 0.01% and about 90%, between about 0.01% and 70%, between about 0.01% and about 50%, between about 0.01% and about 25%, between about 0.01% and about 10%, between about 0.01% and about 5% or between about 0.01% and about 1% of the bulk glucose concentration, a receiver comprising a potentiostat configured to apply a pulsed voltage to the electrochemical biosensor at a pulse period and a pulse width, computer readable memory for receiving and storing sensor response data, a computer processor operably connected to the receiver comprising executable computer code to generate a time series comprising amplitude and frequency data from the sensor response data, and a display for visualizing the obtained sensor response data. In some embodiments, the system can apply a pulsed voltage. In some embodiments wherein the system is applies a pulsed voltage, the pulsed voltage has a pulse period of between about 1 second and about 100 minutes. In some embodiments, the pulse period is between about 5 seconds and about 2 minutes. In some embodiments, the pulse period is between about 5 seconds and about 20 seconds. In some embodiments, the pulse width is between about 10 microseconds and about 100 seconds.

In some embodiments, provided herein is a method of determining a glycemic state of a subject comprising measuring glucose concentration of the subject at time intervals over a period of time to provide glucose concentration data, filtering the glucose concentration data obtained at time intervals over the period of time to provide filtered glucose concentration data; calculating a point to point difference of the filtered glucose concentration data over the period of time to provide a time series, extracting frequency and amplitude information from the time series that is proportional to the change of concentration of glucose, and comparing the frequency and amplitude information to patterns of frequency and amplitude information corresponding to known patterns and thereby establishing the glycemic state of the subject. In some embodiments, the glucose concentration of the subject is measured using a glucose biosensor. In some embodiments the glucose biosensor comprises one or more working electrodes, a glucose responsive sensing layer associated with and in electrical contact with the one or more working electrodes, the sensing layer comprising a glucose-specific enzyme, wherein at least a portion of the sensor is adapted to be subcutaneously positioned in vivo in the subject. In some embodiments the glucose-specific enzyme is glucose oxidase. In some embodiments, the glucose biosensor further comprises a diffusion limiting layer. In some embodiments the time intervals are between about 20 seconds and about 30 minutes, in some embodiments are between the time intervals are between about 20 seconds and about 5 minutes, in some embodiments the time intervals are between about 1 second and about 2 minutes. In some embodiments, the period of time is between about 2 hours and about 24 hours. In some embodiments, the period of time is between about 3 hours and about 24 hours. In some embodiments the period of time is between about 1 hour and about 5 hours. In some embodiments, the period of time is between about 1 hour and about 7 days.

In some embodiments provided herein is a method of determining a metabolic fingerprint of a subject, comprising measuring sensor data responsive to a concentration of an oscillating biological substance over a period of time to provide concentrations of the oscillating biological substance, calculating point to point differences in concentration over the period of time to provide a time series; extracting frequency and amplitude information from the time series that is proportional to change of concentration of the oscillating biological substance, and establishing a metabolic fingerprint of the biological substance for the subject.

In some embodiments, provided herein is a method of diagnosing a state of glycemia in a mammal with an electrochemical biosensor for measuring a glucose level comprising a working electrode, a counter electrode, an active zone, and a diffusion limiting barrier separating the working electrode from bulk solution, comprising placing a biosensor in proximal contact with cell surfaces of a subcutaneous region of the mammal, applying a voltage from a potentiostat to the electrochemical biosensor such that the glucose concentration proximal to cell surfaces oscillates between about 0.01% and about 20% of the bulk glucose at the surface region of the working electrode. In some embodiments, the mammal is a human. In some embodiments, the glucose level is a concentration. In some embodiments, the cell surface is selected from the cell surface of fibroblasts, adipocytes, or myocytes.

In some embodiments, provided herein is a method of diagnosing a state of glycemia in a subject comprising obtaining glucose sensor response data from a subcutaneous glucose sensor, converting the data to a time series pattern consisting of changes in sensor response from point to point, and comparing the time series pattern to characteristic patterns of glycemic disease states, glycemic pre-disease states, and glycemic normal states to assess the state of glycemia in the subject. In some embodiments, the pattern is an amplitude pattern. In some embodiments the pattern is a frequency pattern. In some embodiments, converting the data to a time series pattern comprises filtering the data to extract low frequency components. In some embodiments, filtering the data comprises using a wavelet technique. In some embodiments, the wavelet technique is a Haar transform. In some embodiments, filtering the data comprises using a low pass filter. In some embodiments filtering the data comprises carrying out a Fourier transform or spectral density analysis. In some embodiments, low frequency components are those with frequency less than about 0.02 Hz. In some embodiments, the low frequency components are those with frequency less than about 0.01 Hz. In some embodiments, the low frequency components are those with frequency less than about 0.005 Hz. In some embodiments, low frequency components are those less than the pulse frequency. In some embodiments, extracting the low frequency component comprises removing the high frequency components. In some embodiments, the subject is a mammal. In some embodiments, the subject is a human.

In some embodiments provided herein is a method of diagnosing a state of glycemia from a pattern of glucose oscillations in a subject comprising inserting a biosensor within the dermis of the subject, applying energy to the biosensor, recording and storing raw output response data from the biosensor for a period of time, filtering the raw output response data to provide filtered response data, calibrating the filtered response data versus glucose, obtaining periodic sensor response data corresponding to concentrations of glucose in the subject over the period of time, converting the periodic sensor response data to a time series pattern consisting of changes in sensor response from point to point, and comparing the time series pattern to characteristic patterns of different states of glycemia.

In some embodiments the raw output response data is a current response. In some embodiments, the period of time is between about 2 hours and about 24 hours. In some embodiments, the period of time is between about 3 hours and about 24 hours. In some embodiments, the period of time is between about 1 hour and about 5 hours. In some embodiments, the period of time is between about 1 hour and about 30 days. In some embodiments, the time series analysis is wavelet analysis. In some embodiments, the wavelet analysis is a Haar transform. In some embodiments, the subject is a mammal. In some embodiments, the mammal is a human.

In some embodiments, the state of glycemia is type 1 diabetes. In some embodiments the state of glycemia is type 2 diabetes. In some embodiments the state of glycemia is impaired glucose tolerance. In some embodiments the state of glycemia is that within a normal range. In some embodiments the state of glycemia is pre-diabetic. In some embodiments the state of glycemia is metabolic syndrome. In some embodiments the energy is voltage. In some embodiments the energy is pulsed voltage. In some embodiments the characteristic pattern is an amplitude pattern. In some embodiments the amplitude pattern is a point to point difference in glucose over the period of time. In some embodiments the characteristic pattern is a frequency pattern. In some embodiments the frequency pattern is a peak to peak time difference in glucose over the period of time. In some embodiments the characteristic pattern is an amplitude pattern and a frequency pattern. In some embodiments filtering the raw output response data using time series analysis to provide filtered response data comprises using a wavelet technique. In some embodiments the wavelet technique is a Haar transform. In some embodiments filtering the raw output response data using time series analysis to provide filtered response data comprises using a low pass filter. In some embodiments filtering the raw output response data using time series analysis to provide filtered response data comprises carrying out a Fourier transform or spectral density analysis. In some embodiments filtering the raw output response data using time series analysis to provide filtered response data comprises filtering the data to extract low frequency components. In some embodiments the low frequency components are those with frequency less than 0.2 Hz. In some embodiments the low frequency components are those with frequency less than 0.02 Hz. In some embodiments the low frequency components are those with frequency less than 0.01 Hz. In some embodiments the low frequency components are those with frequency less than 0.005 Hz. In some embodiments extracting the low frequency component comprises removing the high frequency components.

In some embodiments, provided is a method of analyzing cellular metabolic oscillations of a subject comprising inserting a biosensor within the dermis of the subject, applying energy to the biosensor, recording and storing raw output response data from the biosensor for a period of time, filtering the raw output response data to provide filtered response data, calibrating the filtered response data versus an oscillating cellular substance versus time utilizing the first or second current differences and a concentration of a reference sample from a fingerstick or venous blood draw wherein the concentration of the substance is determined using an in vitro method of measurement, storing the calibrated filtered response data versus time, detrending the calibrated filtered data by taking a first or second order difference to obtain a series of peaks and valleys versus time, and measuring the time period between consecutive peaks. In some embodiments, the oscillating cellular substance is glucose. In some embodiments, the period of time is between about 2 hours and about 24 hours. In some embodiments, the period of time is between about 3 hours and about 24 hours. In some embodiments, the period of time is between about 1 hour and about 5 hours. In some embodiments, the period of time is between about 1 hour and about 30 days. In some embodiments, the method further comprises calculating an average and standard deviation of the frequency for all peaks within a time series over a period of time. In some embodiments, the method further comprises, calculating the average and standard deviation of peak amplitudes within a time series over a period of time. In some embodiments, the method further comprises determining the mean and standard deviation of bulk glucose concentrations corresponding to the peak amplitudes in a time series. In some embodiments, the method further comprises determining mean and standard deviation of glucose values corresponding to peak amplitudes in dG₁ or dG₂ (first and second differences in glucose). In some embodiments, the method further comprising, using dG₁ & dG₂ peak amplitudes to calculate the rate of change (velocity) & the rate of change of the rate of change (acceleration). In some embodiments, the method further comprises integrating the area under the peaks in a time series as a current or glucose concentration. In some embodiments, the method further comprises calculating a normalized composite score for the glucose response data over a period of time based on measured parameters of peak amplitude, peak area, peak frequency and bulk glucose concentration. In some embodiments, the method further comprises comparing the normalized composite score to a data base of normalized composite scores from subjects with normal glucose levels, those with pre-diabetes, metabolic syndrome, type 1 diabetes, or type 2 diabetes to determine a state of glycemia. In some embodiments, the method further comprises determining where the calculated composite score fits within the spectrum of diabetes from normal to impaired glucose tolerance (pre-diabetes) to the metabolic syndrome to type 1 or type 2 diabetes in order to provide diagnostic criteria of a state of glycemia.

In some embodiments provided herein is a method of determining a glycemic state of a subject comprising measuring glucose concentration of a subject at time intervals over a period of time using a continuous glucose monitor (CGM) to provide glucose concentration data, filtering the glucose concentration data from the continuous glucose monitor obtained at time intervals over the period of time to provide filtered glucose concentration data; calculating a point to point difference of the filtered glucose concentration data over the period of time to provide a time series, extracting frequency and amplitude information from the time series that is proportional to the change of concentration of glucose, and comparing the frequency and amplitude information to patterns of frequency and amplitude information corresponding to known patterns and thereby establishing the glycemic state of the subject.

In some embodiments provided herein is a method of calibrating an analyte sensor that is operably connected to an electronic receiver and subcutaneously inserted in a subject the method comprising receiving sensor response data from the analyte sensor that is operably connected to an electronic receiver and subcutaneously inserted in a subject to provide analyte sensor response data, filtering the analyte sensor response data, calculating the point-to-point difference in the analyte sensor response data obtaining an ex vivo, post subcutaneously inserted, calibration value from an analyzed sample of a body fluid of the subject, inputting the ex vivo, post subcutaneously inserted, calibration value from an analyzed sample of a body fluid of the subject into the transceiver thereby creating a time stamped analyte value, calibrating the subcutaneously inserted analyte sensor using the time stamped analyte value and the time corresponding point-to-point difference in the implanted sensor response to provide calibrated sensor response data, and transmitting the calibrated sensor response data to a receiver for visual display of concentration versus time. In some embodiments, the analyte sensor is a glucose sensor. In some embodiments the filtering includes a moving average filter. In some embodiments the filtering includes a low pass filter. In some embodiments the filtering includes a wavelet. In some embodiments the point-to-point difference is a first difference. In some embodiments the point-to-point difference is a second difference. In some embodiments, obtaining an ex vivo, post subcutaneously inserted calibration value is obtained from an analyzed sample of a subject's blood. In some embodiments, the post subcutaneously inserted calibration value is obtained using a blood glucose meter. In some embodiments, the post subcutaneously inserted calibration sample is measured using a laboratory reference method.

In some embodiments provided herein is a method of calibrating an analyte sensor for in vivo use comprising manufacturing a batch of sensors; obtaining a subset of the manufactured batch of sensors, measuring the response of each sensor in the subset of manufactured sensors in an in vitro solution with varying concentrations of the analyte and obtaining slope and intercept data for each sensor in the subset of sensors, averaging the individual calibration data from the subset of sensors to obtain batch calibration data, encoding the batch calibration data onto the remaining sensors in the batch, and entering the encoded batch calibration data into a subject's analyte transceiver at the point of use to provide calibrated in vivo sensors. In some embodiments the entering of the encoded batch calibration data is performed manually. In some embodiments, the entering of the encoded batch calibration data is performed optically. In some embodiments, the entering of the encoded batch calibration data is performed wirelessly.

In some embodiments, provided herein is a device for measuring metabolic oscillations of a subject comprising a sensor module which comprises a sensor, configured for mounting on skin, for determining a level of a metabolite over a period of time to provide response data, a means for deploying the sensor into the skin of a subject, a wireless transceiver for sending and receiving data operably connected to the sensor comprising computer readable memory configured for receiving and storing the response data, and a computer processor operably connected to the sensor module comprising executable computer code to obtain a time series comprising amplitude and frequency data from the response data, and a means for extracting metabolic oscillations from the time series comprising amplitude and frequency data from the response data.

In some embodiments, the device further comprising a hand-held monitor comprising a wireless transceiver to send and receive data, a keypad, a display, and a computer processor operably connected to the hand-held monitor comprising executable computer code to store and display data, calibrate the sensor response data and wirelessly connect to the sensor module. In some embodiments the metabolic oscillations are glucose oscillations. In some embodiments the device is configured to display changes of the concentration of glucose over time. In some embodiments, the device further comprises a display, and in yet other embodiments, the display is configured to show in graphical form response data, concentration, or metabolic patterns. In some embodiments, the device further comprises a mounting unit for mounting on a skin of the subject. In some embodiments, the subject is human.

In some embodiments, the device further comprises an electrochemical cell comprising a working electrode and a counter electrode, a voltage source which provides a voltage between the working electrode and the counter electrode when electrically connected through a conductive medium, and a computing device which measures the dynamic current output from the working electrode. In some embodiments, the working electrode is coated with a protein layer and a diffusion limiting barrier covering the protein layer. In some embodiments, the voltage source is a potentiostat, and in yet other embodiments, the device is configured or programmed such that the potentiostat can apply voltages at time intervals under the control of the operator of the device. In some embodiments, the counter electrode is in contact with a diffusion limiting barrier. In some embodiments, the device is configured such that a voltage waveform is applied between the counter electrode and working electrode. In some embodiments, the device further comprising a reference electrode. In some embodiments, the electrochemical cell comprises an active zone, the active zone comprising glucose concentration between about 0% and about 50% of the bulk glucose concentration during potentiostat voltage pulses. In some embodiments, the counter electrode diffusion limiting barrier can be the skin of the subject.

In some embodiments, the diffusion limiting barrier comprises a polymeric material and in other some embodiments the polymeric material comprises a polyurethane. In some embodiments, the protein is glucose oxidase.

In some embodiments of the device, the means for extracting cellular glucose oscillations from the time series comprising amplitude and frequency data from the response data is a filter. In some embodiments, the filter is a wavelet, a moving average filter, a low pass filter, or a recursive filter. In some embodiments, the response data is an analog signal. In some embodiments of the device, obtaining a time series comprises computing a point to point difference in sensor response versus time. In some embodiments, the signal is a voltage, a current, optical, or electromagnetic energy. In some embodiments, the device comprises a sensor coated with immobilized living cells selected from eukaryotic cells, prokaryotic cells, yeast cells, mammalian cells, cancer cells or combinations thereof. In some embodiments, the sensor is a biosensor. In some embodiment, taken together, the sensor, the transceiver, hand-held monitor and the computer processor are a continuous glucose monitor (CGM) or are incorporated in a continuous glucose monitor (CGM).

In some embodiments, methods and systems are provided herein for continuously measuring cellular metabolic patterns and processing the data from these measurements to provide a pattern or “metabolic fingerprint” of biological processes such as glucose metabolism and storing and analyzing the metabolic data. The resulting information can be utilized to determine whether there are aberrations in the patterns of metabolism that may be diagnostic of certain metabolic diseases such as diabetes or the Metabolic Syndrome.

Of the many advantages of the various embodiments described herein the methods, systems, and devices provided can easily diagnose the early stages of metabolic disease before there are overt symptoms without the necessity of frequent blood draws. Another advantage is the ability to measure the bulk concentration of an analyte along with its cellular metabolic component yielding information that may be used to characterize a glycemic state or to control an infusion device such as an insulin pump.

### BRIEF DESCRIPTION OF THE DRAWINGS

FIG. 1 shows a partial schematic of a potentiostat useful in the invention that can be used to apply a steady-state or pulsed voltage between a working electrode (WE) and counter electrode (CE) of an electrochemical cell. A reference electrode is depicted as RE;

FIG. 2 shows a three-electrode electrochemical cell with three electrodes, (C) counter, (W) working and (Ref) for a reference electrode held in a conductive medium;

FIG. 3 illustrates an example of an electrochemical cell wherein the working electrode (W) is coated with a protein layer, such as glucose oxidase (GOx) and albumin. In addition, the protein layer is covered by a second layer including a diffusion limiting barrier composed of a polymer such as polyurethane;

FIG. 4 is a view of a protein layer, on a working electrode, containing glucose oxidase covered with a diffusion limiting barrier on a working electrode versus a counter electrode on the right side of the drawing. The reactions occurring within the active zone of the working electrode also are shown for the reaction of glucose with glucose oxidase (GOx). The oxidation of hydrogen peroxide produced from the oxidation of FADH₂ produces oxygen and a current proportional to glucose concentration. These reactions are depicted in equations 4-8;

FIG. 5 is a depiction of a working electrode having a protein layer encapsulated within a diffusion limiting barrier versus a counter electrode covered with a diffusion limiting barrier. The resistance and capacitance contributions to the impedance between the working electrode surface and the counter electrode surface are shown along with other descriptive terms, where Rw is the intrinsic resistance of the working electrode, C_{dl} is the capacitance of the double layer, R_{WZ} is the resistance of the fluid within the Active Zone, R_{MW} is the resistance across the diffusion limiting barrier, RE is the resistance of the bulk fluid surrounding working and counter electrodes, ISF or interstitial fluid is an example of a bulk fluid, R_{MC} is the resistance across a diffusion limiting barrier over the counter electrode, R_{CZ} is the resistance of the active zone around the counter electrode and Rc is the intrinsic resistance of the counter electrode;

FIG. 6 shows a series (400) of intermittent square wave voltage pulses, progressing through time, having a total pulse period (τₜ) equal to the sum of a voltage on-time (τ₁) and a voltage off-time. (τ₂). A vertical rectangular box shows a rising voltage or current (410), maximum voltage (E_{wr}) or maximum current, (Ip) (420), decaying current vs. time transient (iₜ) 430, the end of on-time period (τ₁) and falling voltage are defined by (440). The area defined by the on-time line on the X-axis, vertical solid line (410), horizontal solid line labeled E_{wr} and vertical solid line (440) delineate the square wave voltage pulse, the on-time period (τ₁) and decaying current vs. time transient (430). The line (440) shows a rapid fall-off in the working electrode voltage at the end of the pulse period (τₜ);

FIG. 7 shows a flow chart of how the time series data is obtained and utilized comprising steps 1-10a,b;

FIG. 8 shows an example of the measurement of in vitro working electrode response time (1.25 min) derived from the addition of glucose to an electrochemical cell having an amperometric glucose oxidase working electrode in pH 7.4 PBS;

FIG. 9 shows discrete, stepped responses from the serial addition of glucose to an in vitro electrochemical cell having an amperometric glucose oxidase working electrode in pH 7.4 PBS;

FIG. 10 illustrates (100), the in vivo response of an intradermal, amperometric glucose oxidase biosensor (120), implanted within the skin of a swine, to a bolus injection of glucose at approximately 180 minutes. The working electrode response exhibits a continuous trace between approximately 180 minutes and 280 minutes without the discrete steps shown in FIG. 9. The open circles with 20% error bars (100) represent reference glucose measurements obtained with a YSI (Yellow Springs Instruments) glucose analyzer;

FIG. 11 shows an example of an in vivo configuration for an implanted three-electrode cell (300) including working electrode (W), a counter electrode (C), a reference electrode (R), a skin surface (310), a skin thickness (315), subcutaneous tissue and interstitial fluid (320), an active zone (325), a diffusion limiting barrier (330), resistance (Rs) between the working and counter electrodes and uncompensated resistance (Rᵤ) between the working and reference electrodes;

FIG. 12 shows a graph of a series of square-wave voltage pulses, {[E_{wr}]₁}ₙ, each having a defined pulse width period [τ₁]ₙ, an inter-pulse period [τ₂]ₙ and current transients, [iⱼ]ₙ resulting from its application to the working electrode of a 3-electrode electrochemical cell;

FIG. 13 depicts a scheme for a CGM glucose processing system 12 that applies a voltage pulse to the biosensor using a waveform generator 20, current sampling system 22 generating sampled currents 36 [iⱼ]ₙ, bioanalysis system 24 records the biosensor responses, calculation system 32 computes analyte concentrations and using output system 34 displays the results on a hand-held display 38;

FIG. 14 shows the response versus time for a glucose sensor having yeast cells immobilized on its surface showing unsynchronized vs synchronized glucose oscillations caused by oscillating glucose metabolism within the immobilized yeast cells;

FIG. 15A is an expanded view of FIG. 14 from 650 min to 850 min showing clusters of synchronized cells exhibiting entrained cellular glucose oscillations in yeast cells immobilized on a glucose biosensor;

FIG. 15B is filtered and smoothed data from FIG. 15A. The bulk concentration was about 200 mg/dL. The period of the oscillations was 5.8±1.5 min with an average amplitude of the peaks was ±75 mg/dL which was about 38% of the bulk glucose concentration;

FIG. 16 shows a cartoon depicting how the yeast cells are immobilized within a hydrophilic membrane layer on the surface of a glucose biosensor. The glucose from the bulk solution, G_{b}, diffuses into the layer of yeast cells where a fraction, Gₘ, is metabolized by the yeast cells giving rise to an oscillating flux of glucose, Gₑ, in the extracellular fluid surrounding the yeast cells. The Gₑ glucose diffuses into the enzyme layer where it is oxidized by glucose oxidase (GOx). The hydrogen peroxide produced from the oxidation of FADH₂ within the GOx enzyme, by dissolved oxygen, is oxidized by the platinum electrode to produce a current, protons and an oxygen molecule. Since Ge is oscillating, the sensor response at the platinum (Pt) electrode also oscillates;

FIG. 17 shows the steps of metabolic cellular glycolysis in yeast and mammals. The vertical metabolic pathway consists of the same steps for both yeast and mammals. Eukaryotic cells including mammalian cells and yeast cells give rise to characteristic glucose oscillations;

FIG. 18 shows the basis of the Haar wavelet as a “lifting scheme” consisting of two parts, one is an average or low frequency component, e.g. average glucose concentration or average sensor response and the second is a difference in response or high frequency component. The high frequency component contains most of the noise. At each stage (Hₙ) of the averaging and differencing process, the initial signal can be re-constructed using the averages and differences in the reverse order. Each averaging & differencing step is designated by steps Hₙ where n=(1, 2, 3 . . . n). The Harr transform contains both low frequency (averages) and high frequency (differences) components. The low frequency (average) component can be used to derive the first and second differences;

FIG. 19A is a graph of the averaged Haar level H₇ bulk glucose concentration (gray trace) on the left hand Y-axis versus the corresponding second (dG2) point-to-point differences of H₇ glucose concentrations on the right hand Y-axis (black trace) for a subject with normal glucose metabolism, where the X-axis is time in FIGS. 19A,19B & 19C; FIG. 19B is a graph of the averaged (H₇) bulk glucose concentration (gray trace) on the left hand Y-axis versus the corresponding second (dG₂) point-to-point differences of H₇ glucose concentrations on the right hand Y-axis (black trace) for a subject with type 2 diabetes, FIG. 19C is a graph of the averaged (H₇) bulk glucose concentration (gray trace) on the left hand Y-axis versus the corresponding second (dG₂) point-to-point differences of H₇ glucose concentrations on the right hand Y-axis (black trace) for a subject with type 1 diabetes;

FIG. 20 is a depiction (200) of how glucose oscillations at a constant or steady state glucose concentration 210 (see FIGS. 15A & 15B) can be observed directly and where a first difference (220) dG₁ (glucose) or di₁ (sensor response current) may be used to detrend the data and characterize the glucose oscillations; and

FIG. 21 is a depiction, 500, of how glucose oscillations at non-steady state glucose concentrations, 510, may occur superimposed on the bulk glucose concentration, 520, within the interstitial fluid of a mammal. In order to extract the oscillations data, 510, both a first and second difference may be necessary as shown in FIG. 19A, FIG. 19B and FIG. 19C.

### DETAILED DESCRIPTION

### Definitions

As used herein and in the appended claims, the singular indefinite forms “a”, “an”, and the singular definite form, “the”, include plural referents unless the context clearly dictates otherwise. Thus, for example, reference to a current transient includes a plurality of such current transients and reference to an analyte includes reference to one or more analytes and equivalents thereof known to those skilled in the art, and so forth.

As used herein, the term “about” is defined as an approximation of a value that is within plus or minus 30% of the actual or average value, or as understood by persons of skill in the art, any numerical values or ranges that provide suitable dimensional tolerance. As used herein, the terms “subject,” “host,” “user,” and “patient” either as explicitly recited or a recited reference, refer to any human or animal subject and are not intended to limit the systems or methods to human use.

As used herein, the term “sensor-cellular interface” refers to a condition wherein a subcutaneous sensor is in proximate contact with cells of subcutaneous tissue, for example fibroblasts within the dermis. Proximate contact is defined as the sensor surface being within a certain distance, within ≤2 mm, of a cell surface in order to sense the flux of an analyte at the cellular surface.

“Cellular interface” is defined as the outside surface of a cell. It can also mean the volume of aqueous fluid within the extracellular environment between the outer surface of a sensor and a cell surface.

In some embodiments, the means for extracting cellular metabolic oscillations from the time series comprising amplitude and frequency data from the response data is a filter. As used herein, the term filter, filtering, or noise reduction is defined, and understood by those skilled in the art of data analysis as methods of removing noise or unwanted data from an analog or digital data stream using either mathematical or electronic means. Filters that can be used in various embodiments include low pass filters (removes high frequencies), high pass filters (remove low frequencies), linear or non-linear filters, simple averaging filters, weighted averaging filters, moving average filters, exponential average filters, polynomial filters, least square filters, smoothing splines, kernel smoothing, local regression filtering, Kolmogorov-Zurbenko filtering, Laplacian smoothing, Ramer-Douglas-Peucker algorithms, Savitzky-Golay smoothing, stretched grid smoothing or combinations thereof. Other filters include, for example, wavelet filters, statistical filters, fast Fourier transform filters, additive smoothing filters, Butterworth filters, Kalman filter, and recursive filters. An example of a wavelet filter is a filter that uses a Haar transform.

As used herein, noise can be defined or understood by those skilled in the art as random noise, white noise with no coherence or coherent noise introduced by how a device's mechanism or processing algorithms function.

As used herein, the term “voltage application” is defined as a voltage applied between the working and counter electrodes of an electrochemical cell. The term voltage application can be used to describe a steady state voltage, a stepped voltage, or a pulsed voltage. A steady state voltage, Vs, as used herein is wherein a constant or fixed voltage is applied between the counter and working electrodes for an indefinite period of time tₛ. A stepped voltage as used herein is defined as a voltage V₁ applied for a time t₁ followed by a second voltage V₂ applied for a time t₂ or a series of different voltages applied for any period of time between the counter and working electrodes. A stepped voltage can be applied once or multiple times for an indefinite period. A pulsed voltage, as used herein, consists of a voltage waveform applied for a period of time t₁, followed by a voltage off period of time t₂ that can be repeated through time at regular intervals. It is understood by persons of skill in the art that a “voltage application” can include a voltage pulse, a steady state voltage, a stepped voltage, or combinations thereof. The voltage waveform can consist of a square wave, triangular wave or any other waveform.

The systems, methods, and devices, as provided herein in various embodiments, can be used with continuous glucose monitors (CGM) known in the art. For example, U.S. Pat. Nos. 7,399,277; 7,651,489; 8,217,946; 8,521,558; 8,972,196; 7,615,007; 7,976,492; 8,005,524; 8,460,231; 8,562,558; 9,041,730; 7,920,907; 8,211,364; 8,475,732; 8,591,410; 8,798,934; and 8,924,159, describe continuous glucose monitors that can be used in various embodiments of the systems, methods, and devices provided by this disclosure. U.S. Pat. Nos. 7,399,277; 7,651,489; 8,217,946; 8,521,558; 8,972,196; 7,615,007; 7,976,492; 8,005,524; 8,460,231; 8,562,558; 9,041,730; 7,920,907; 8,211,364; 8,475,732; 8,591,410; 8,798,934; and 8,924,159 are hereby incorporated herein by reference. Persons of skill in the art would understand the general applicability of known continuous glucose monitors to the systems, methods, and devices as provided herein such that persons of skill in the art could adapt the methods and systems taught by this disclosure to other known continuous glucose monitors.

Persons of skill in the art would understand that a variety of biosensors are compatible with the systems, methods, and devices disclosed herein. Glucose biosensors have been known for some time and the systems, methods, and devices taught by this disclosure are not dependent on any particular glucose biosensor being used. For example, the glucose biosensors taught in U.S. Patent publication Nos. 2007/0299617 and 2010/0213079, to Willis, can be used with the methods, systems, and devices disclosed herein. U.S. Patent publication Nos. 2007/0299617 and 2010/0213079 are hereby incorporated herein by reference. Other examples of compatible glucose biosensors are taught in U.S. Pat. Nos. 6,893,552; 7,064,103; 7,368,190; 7,462,264; 7,695,608; 7,955,483; 8,280,476; 8,568,578 and 8,715,981 which are hereby incorporated herein by reference.

The term “computing system” as used herein means a system comprising a micro-processor, an input device coupled to the micro-processor, an output device coupled to the micro-processor, and memory devices coupled to the micro-processor. The input device can be, a touchpad or a miniature keyboard, etc. The output device can be a printer, a plotter, a computer screen, a wireless data transmitter, a data transmission cable (e.g., a USB cable) etc. The memory devices can be dynamic random access memory (DRAM), random access memory (RAM), or read-only memory (ROM), and the like. The memory device includes computer code. For example, the computer code can be used for collecting and storing data, time series algorithms and calibration algorithms. The micro-processor executes the computer code. In some embodiments, the memory device includes input data and the input data includes input required by the computer code. The output device, in various embodiments, displays output from the computer code. Memory devices can be used as a computer readable medium (or a computer readable medium or a program storage device) having a computer readable program code embodied therein and/or having other data stored therein, wherein the computer readable program code comprises the computer code. A computer program product (or, alternatively, an article of manufacture) of the computer system can comprise the computer usable medium or the program storage device. Any configuration of hardware and software, as would be known to a person of ordinary skill in the art, can be utilized to configure the computer system.

A “peak finding algorithm” is a method for measuring the peaks within a time series. The data can be filtered using a moving average filter or some other filter that removes high frequency noise, for example a Harr Transform. An example of a peak finder algorithm includes computing the local or entire average and standard deviation of smoothed data. Values which are larger than ±x times (e.g. 1-2 SDs) the standard deviation are considered peaks having an amplitude value and time value. Another way to determine peak data is by using commercially available software such as PeakFit from Systat Corp and other commercially available software for time series analysis.

The term “sensitivity (S)” as used herein is defined as the change in the response of the biosensor per unit change in concentration of an analyte. In the case of a glucose oxidase (“GOx”) amperometric enzyme biosensor, the biosensor response current is directly proportional to the glucose concentration. Sensitivity S is expressed as the change in biosensor response current per unit of change in concentration, e.g. nA/mg/dL or nA/mM, where mM is an abbreviation for millimolar (millimoles/Liter) or (mmol L⁻¹) and nA is an abbreviation for nanoamps. The sensitivity may be determined by linear regression of the biosensor response current v. analyte concentration. The slope of such a plot is the sensitivity S.

A “biosensor” can defined as a single electrode or a combination of electrodes that includes a counter electrode and/or a reference electrode thereby constituting an electrochemical cell.

“Equilibration period”, “equilibration time”, or “break-in period”: When a biosensor is implanted within a subject or used in vitro within a test cell, a period is generally required, in most embodiments, for equilibration of the biosensor's response to the conductive fluid surrounding the implanted biosensor. The period for the biosensor's response to reach its steady-state value is called the equilibration period. The terms “equilibration time” and “break-in period” are also used by person of skill in the art and are synonymous with the term equilibration period.

“Biosensor equilibration time”, as indicated above, when one or more electrodes of an electrochemical biosensor are implanted within a subject or used in vitro within a test cell, a period is required for equilibration of the biosensor's response current to the conductive fluid surrounding the biosensor. The time required for the sensor current to reach its steady-state value is called the equilibration period. An equilibration period exists even in the absence of target analyte. The equilibration time is a function, inter alia, of the thickness and chemical complexity of the catalytic surface (sensing element) of the working electrode. For example, if the enzyme layer that forms the catalytic surface of the working electrode is relatively thin, the equilibration time may be less than 30 minutes. If however, the enzyme layer that forms the catalytic surface of the working electrode is relatively thick or covered with non-enzymatic materials, such as polymers or proteins, then the equilibration time may be greater than 30 minutes, approaching hours. In either case a maximum response current is initially observed that gradually decreases over time to a steady state value consistent with the quantity of the target analyte being measured.

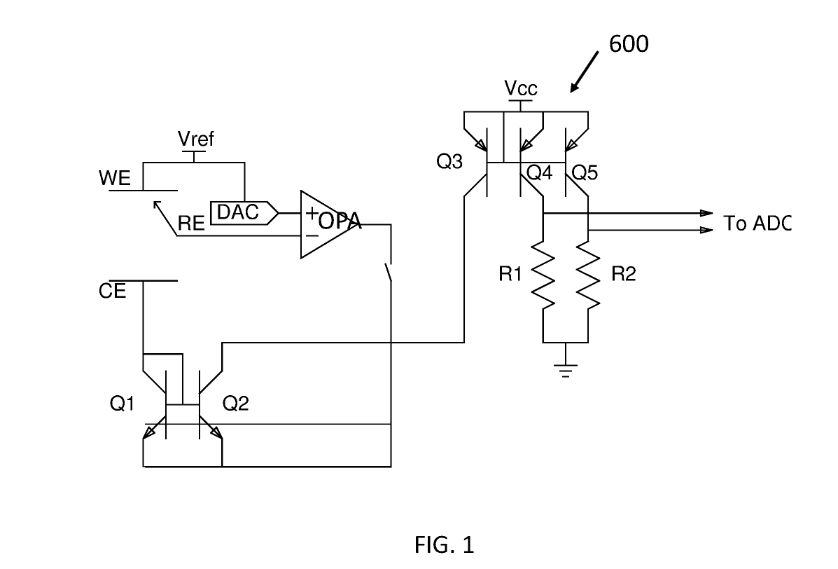

As shown in FIG. 1, a “potentiostat” (600) is used to supply a voltage between the working (w) and counter (c) electrodes. By means of a feedback circuit, the potentiostat varies the applied potential between the working and counter electrodes (E_{wc}) to maintain a constant potential between the reference and working electrodes.

The term “diffusion limiting barrier” refers to a covering over a working electrode or counter electrode including a porous or semi permeable material, such as, for example, a polymeric material that limits diffusion of species into and out of the working electrode active zone. The diffusion limiting barrier also prevents migration of chemical species out of the biosensor, such as, for example, enzymes and mediators, or it may prevent the migration of unwanted components within tissue, cells or body fluid into the biosensor active zone, wherein, in either case, they may adversely affect the biosensor's response. The diffusion limiting barrier may also serve to limit the diffusion of a target analyte into the active zone, thus improving the linearity of the biosensor's response, or preventing saturation of the response. The terms “membrane,” “semi permeable material,” “semi permeable membrane,” “coating,” “barrier,” “protective barrier,” “diffusion limiting barrier,” “diffusion limiting coating,” or “barrier membrane” are generally understood to be synonymous herein. Other definitions are described in the specification and defined within the context of their use.

This technology relates to devices and methods for measuring analytes dissolved in a conductive medium. The working electrode can in some embodiments be covered with a diffusion limiting barrier. An interface or space between the underside of a diffusion limiting barrier and a working electrode can create a small, confined volume (active zone) where concentrations of ions, charge carrying by-products or conductivity enhancing species from chemical reactions and/or electrochemical oxidation or reduction of an analyte can change the conductance within the active zone of a working electrode. The diffusion limiting barrier can temporarily limit the flow of ionic or conductivity enhancing species away from the working electrode active zone across a diffusion limiting barrier into the bulk solution on the outside surface of a working electrode.

In general, provided herein for use with various embodiments described in this application is an electrochemical cell including a plurality of electrodes; and a computing system that determines and provides a read-out of concentration of an analyte or analytes. This technology relates to measurements of dissolved analytes contained within a conductive medium or the interaction of a conductive medium, containing an analyte, with an electrical conductor. Some embodiments described herein include an electrochemical cell. The minimum requirement for an electrochemical cell is that it has at least two electrodes defined as a working electrode (e.g., anode or cathode) and a counter electrode (e.g. cathode or anode) with a conductive medium between the two electrodes to allow completion of an electrical circuit. The working electrode is defined as the electrode at which electrochemical oxidation or reduction may occur to produce a response in the form of a current, voltage or time that is proportional to analyte concentration. If the working electrode is the positive terminal (anode) of the cell, the counter electrode is the negative terminal (cathode) and vice versa.

The electrochemical cell, as used in various embodiments, can be interfaced to a system, including for example, a potentiostat for application of a voltage or current between the counter electrode and working electrode. The system can also include a computing or recording system that: (1) records input and output of the electrochemical cell, (2) stores data in memory, (3) performs calculations on the data and (4) visually displays the data or calculations on the data or analyte concentration. To obtain an unknown analyte concentration from a sensor response measurement an equation is used that relates a response to analyte concentration. Most of the calculations described herein are linear and can be defined by the simple equation: y=mx+b; wherein y denotes the response, m is the slope of response versus analyte concentration, x is analyte concentration and b is the y-intercept of a plot of response versus analyte concentration. These plots are sometimes referred to as calibration plots.

Using the slope and intercept from any of the graphs, an unknown analyte concentration is calculated as follows:

x=(y−b)/m (1)

The slope (m) and intercept (b) from a graph of response on the Y-axis versus analyte concentration on the X-axis are substituted into equation 1 to calculate analyte concentration from a measured response. The slope and intercept may be determined prior to analysis of an unknown analyte concentration by plotting response on the Y-axis versus known concentrations of analyte on the X-axis and using linear regression to determine the slope and intercept, or a line can be drawn between the response points versus analyte concentration which line stops at or crosses the Y-axis, that point on the Y axis yields the intercept, the response at zero analyte concentration, and the difference between at least two response points divided by the corresponding difference in analyte concentration yields the slope.

Another way to calculate analyte concentration is to use a single-point calibration. This requires that at least one concentration that is known prior to the calculation of other unknown analyte concentrations. An example is the continuous measurement of glucose in vivo. It is possible to pre-calibrate (also known as “factory calibration” or “in vitro calibration”) sensors prior to in vivo use by using physiological samples such as buffer, whole blood, plasma, or serum and determining in vitro sensor response to increasing levels of analyte. The slope and intercept values may be entered into the memory of the receiver that is operably connected to an in vivo sensor. To perform an in vivo calibration of an implanted glucose sensor requires an in vivo measurement of glucose using, for example, a blood glucose meter and a sample of blood from a fingerstick or venous blood draw, interstitial fluid (ISF) or other in vivo body fluid. The initial in vivo slope, m₁, from a single fingerstick glucose measurement, [G]₁, from an in vivo sample, can be determined by:

m₁=i₁/[G]₁ (2)

Where i₁ refers to the current response to a known glucose concentration [G]₁ and the subscript 1 or n (n=1, 2, 3 . . . ) indicates the current response and analyte concentration are measured at the same point in time and calibration slopes beyond m₁ are noted as mₙ and a corresponding current response as iₙ. The above equation assumes a zero intercept. In the case of an amperometric enzyme electrode, the response is a measured current and dividing the current by the glucose concentration yields a slope, mₙ and is equal to nanoamps/mM (millimoles/Liter) or nanoamps/mg/dL. Subsequent, unknown in vivo glucose concentrations [G]ₙ may be calculated using the following equation:

[G]n=[G]₁+[(iₙ−i₁)/m₁] (3)

Where the subscript n denotes any glucose measurement taken after [G]₁ and i₁ serves as the intercept. The calibration process can be repeated at any time after measurement of [G]₁ and the new slope, intercept and single point measured glucose concentration used to calculate future unknown glucose concentrations.

The electrochemical cell can be a permanent or an integral part of the system or the electrochemical cell can be a separate unit that plugs into the system. In some embodiments, the electrochemical cell can include a third electrode known as a reference electrode. The system may include a plurality of electrochemical cells, electrodes or an array of electrodes.

Electrodes or combinations of electrodes can be immersed in a conductive medium in which analytes or other species are already present or to which analytes can be added in the form of solids, liquids or gases. The electrodes can be stored in the dry state and later activated by the addition of a conductive medium containing an analyte or the electrochemical cell can be exposed to the air whereby moisture in the air activates the electrodes for the measurement of analytes within air.

The working electrode of the electrochemical cell can have a biological component such as an enzyme, protein, antibody, antigen, RNA, DNA, DNA fragments, synthetic proteins, recombinant proteins, any cells both eukaryotic and prokaryotic or cellular materials, and the like associated with, immobilized, entrapped or near its surface. In such case, the working electrode may be referred to as a biosensor. The biosensor can be used for in vitro or in vivo analyte measurements. An in vivo application can include electrodes or groups of electrodes such as electrochemical cells, wherein all or part are totally or partially in contact with eukaryotic or prokaryotic cells or tissue of humans, animals, or plants. Partially implanted sensor systems can include a plurality of in vivo electrodes with other electrodes ex vivo, as for example, on the skin surface. Together, the in vivo and/or ex vivo electrodes comprise one or more electrochemical cells.

The technology described herein relates to electrochemical cells having at least two conductor(s) that, in combination, can complete an electrical circuit through which a voltage or current can flow. In addition, the cell(s) can contain a plurality of conductors that serve as working, counter, or reference electrodes. In various embodiments described herein, the electrochemical cell includes at least one conductor serving as a working electrode and another conductor, serving as a counter electrode. In some embodiments described herein, the conductors can be held within a fluid medium. The fluid medium can be in direct contact with each conductor or the conductors can be separated by a permeable material, with fluid surrounding both conductors and the permeable material. The permeable material allows the transport of ions and other low molecular weight species, dissolved in the external fluid medium, across the permeable material into the active zone. The fluid medium can be held in place, for example, with an enclosure such as plastic, glass, silicon, ceramic, polymers, adhesives or adhesive pads. The enclosure can also include a conductive gel that surrounds and contacts the conductors. The enclosure can also include body tissue either in vivo or ex vivo. An example of ex vivo tissue is the skin surface and an example of in vivo tissue is any subcutaneous or intradermal tissue.

Suitable conductors for use in various embodiments described herein include noble metals such as platinum, palladium, ruthenium, iridium, alloys such as platinum-ruthenium, platinum-iridium; other metals such as silver, titanium or alloys of metals such as titanium-aluminum, titanium-platinum, titanium-indium-cobalt, nickel alloys such as Inconel, Incoloy or Nitinol and other conductors such as graphite, carbon, glassy carbon, graphene, diamond, diamond-like carbon (DLC), single crystals, forms of carbon such as carbon nano-tubes, Fullerenes, nano-particles, graphene and the like. In addition, conductor materials can be semiconductors such as crystalline or amorphous silicon, doped silicon or other materials such as organic semiconductors. The conductor can also include an inert or non-conductive substrate such as plastic or a ceramic material upon which metal or other conductive materials are deposited by dipping, printing, plating, chemical vapor deposition or other means.

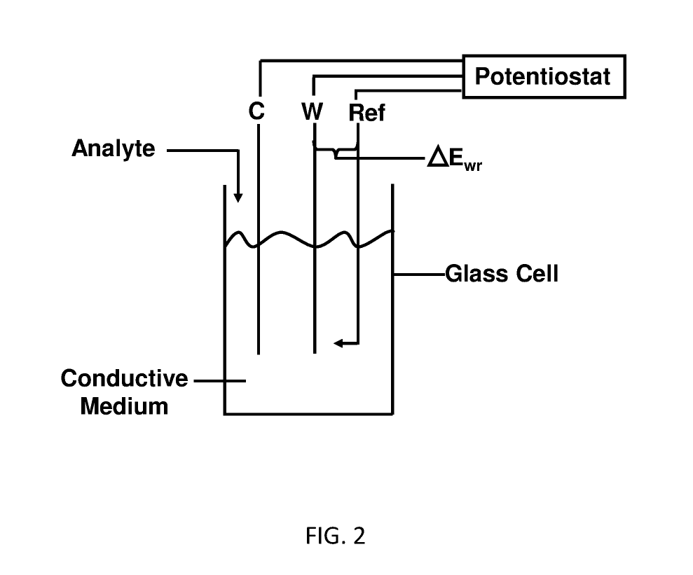

FIG. 2 shows a drawing of a three-electrode electrochemical cell with electrodes immersed in a conductive medium. The three electrodes include a working or sensing electrode, W, having a poise voltage Ewr versus a reference electrode Ref; counter electrode of opposite polarity to working electrode, C, and reference electrode, Ref. For example, the conductive medium may contain dissolved ions derived from electrolytes such as potassium chloride, sodium chloride and/or buffer containing salts. An example of a suitable conductive medium is pH 7.4 phosphate buffered saline (“PBS”) (available from Sigma-Aldrich). An example of a suitable reference electrode is a silver/silver chloride electrode.

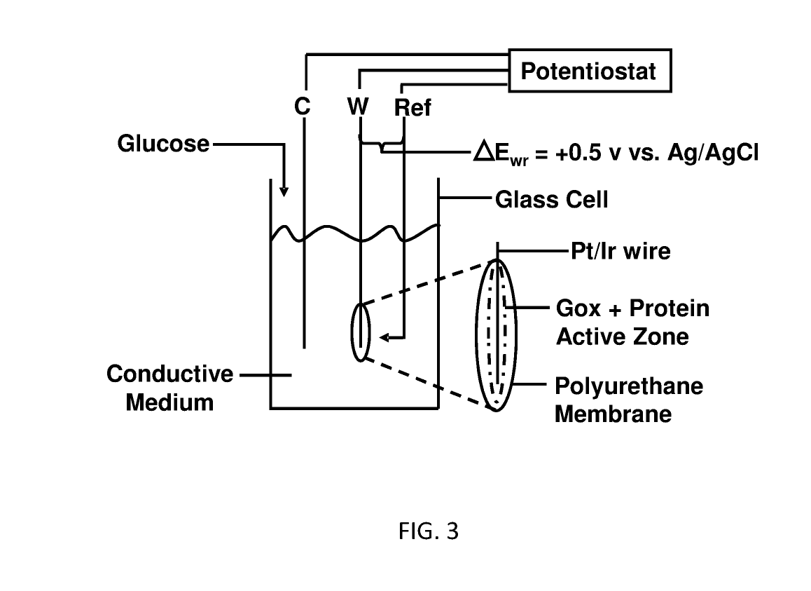

FIG. 3 illustrates an example of an electrochemical cell wherein a working electrode is coated with a protein layer having an active protein and an inert protein, such as glucose oxidase (GOx) and bovine serum albumin (BSA). In addition, the protein layer is covered by a second layer including a diffusion limiting barrier such as a polymer. For example, the polymer may be chosen from any polymer that can be applied by dipping, printing, spraying, spin coating, vapor deposition or in situ polymerization. The diffusion limiting barrier allows the passage of small ionic and low molecular weight molecules such as sodium, potassium, chloride, phosphate, glucose, oxygen, etc., into and out of the active zone but excludes high molecular weight compounds such as proteins or cells.

Examples of suitable polymers are silicones or polyurethanes that can be applied by dipping, printing, spraying or spin coating on the working electrode.

When a working electrode is covered, coated or enclosed within a diffusion limiting barrier, there is a small volume or interface between the inside surface of the diffusion barrier and the surface of the working electrode conductor. This space is referred to herein as the “active zone (AZ)”, where chemical and electrochemical reactions can occur in close proximity to the electrode surface. The active zone may also serve as a vessel divided from the external bulk solution surrounding the working electrode, while still allowing diffusion into and out of the vessel.

The technology described herein in some embodiments relies, in part, on the ability of a diffusion limiting barrier to limit diffusion of products, produced from chemical and electrochemical reactions within an active zone across a diffusion limiting barrier into a conductive medium or bulk solution surrounding the electrode. Products from chemical and electrochemical reactions, occurring within the working electrode active zone, are frequently charged or have high conductivity. The increase in conductivity temporarily reduces the electrical impedance between the working electrode and the counter electrode. These chemical and electrochemical reactions increase the conductance or admittance at the working electrode surface as reflected by the output voltage of an op amp driving a counter electrode.

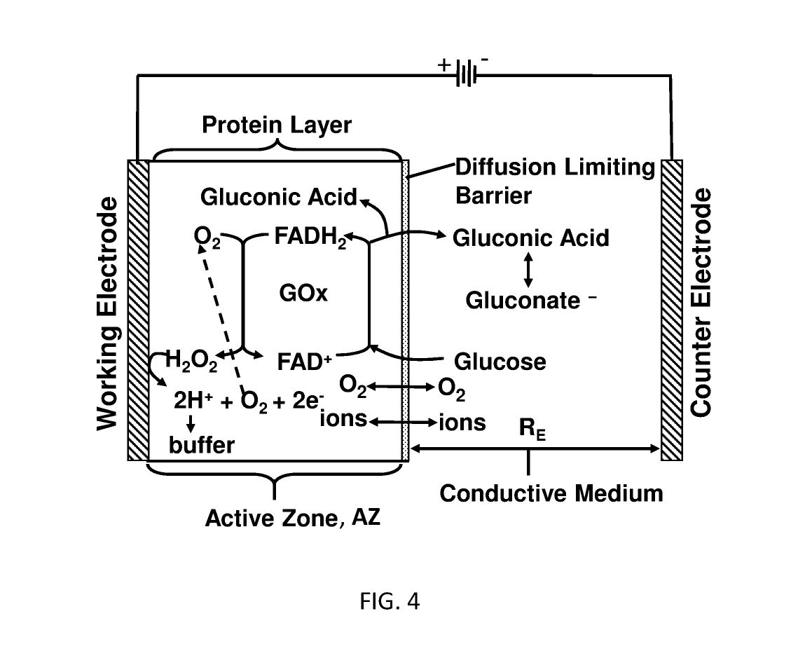

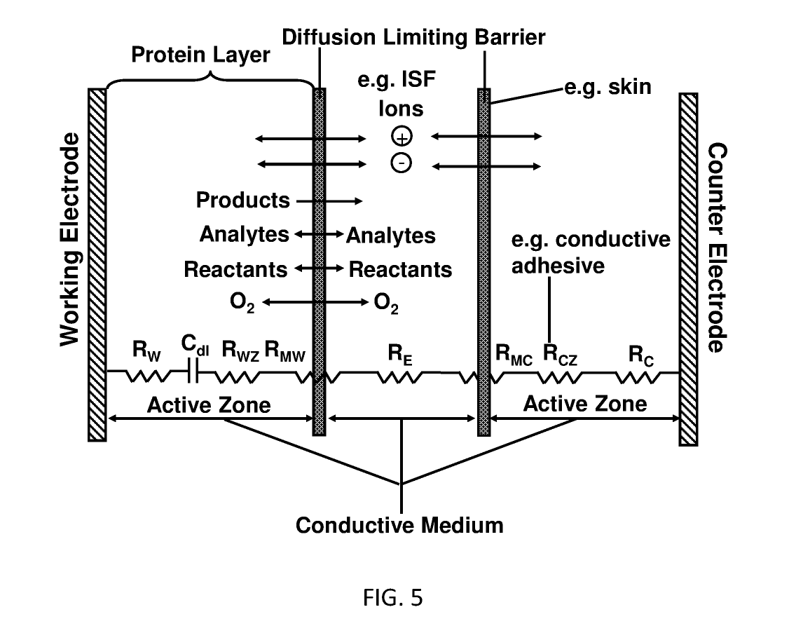

FIG. 4, shows an expanded view (not to scale) of the working electrode shown in FIG. 3. The protein layer, as defined in FIG. 4, can include a mixture of inert proteins, active proteins, or combinations thereof. An example of an active protein is an enzyme such as glucose oxidase (GOₓ). An example of an inert protein is gelatin or albumin. The protein mixture may be cross-linked, for example with glutaraldehyde or another crosslinking agent. The protein layer can be coated with a layer of a diffusion limiting barrier in order to: (a) protect the protein coating on the electrode from the body's immune system; (b) limit diffusion of analytes and unwanted high molecular weight species within the external bulk solution into the active zone or, (c) limit the diffusion of products, proteins or high molecular species, within the active zone, out into the external bulk solution.

FIG. 4 further shows chemical and electrochemical reactions occurring within the active zone of a glucose oxidase working electrode in accordance with some embodiments described herein. Glucose from the external bulk solution diffuses across a diffusion limiting barrier into the active zone where it is oxidized by glucose oxidase (GOₓ) to gluconic acid (or gluconolactone) and the two (Flavin Adenine Dinucleotide) FAD⁺ active sites in GOₓ are reduced to FADH₂. This is followed by oxidation of the FADH₂ groups by dissolved oxygen within the conductive medium and active zone to produce hydrogen peroxide, 2 protons (H+) and oxidized GOₓ. These chemical reactions are catalytic and occur in the absence of an applied voltage. The electrochemical oxidation of hydrogen peroxide yields two protons (2H⁺) and one oxygen molecule (O₂) that can be recycled by the enzyme. The current generated from the electrochemical oxidation of hydrogen peroxide is directly proportional to glucose concentration, the protons produced from the electrochemical oxidation of hydrogen peroxide produce a transient change in the pH, and thus conductance, within the active zone, before being consumed by buffer. The working electrode, active zone, and diffusion limiting barrier together constitute a half-cell of a two-electrode cell. The counter electrode half-cell in the right of FIG. 4 completes an electrical circuit with the working electrode on the left. Besides the working and counter electrodes, a reference electrode can be used to maintain a constant voltage, E_{wr} (FIG. 2, 3), between a reference electrode and working electrode. Referring to FIGS. 4 & 5, the matrix within the active zone may include water, electrolytes, reactants, glucose, oxygen, proteins, glucose oxidase, enzymes, substrates, polymers, aqueous gels, mediators and products, such as hydrogen peroxide, hydrogen peroxide anions, gluconic acid, gluconolactone, anions, gluconate, cations, hydrogen ions (H+), and other low molecular weight species. The diffusion limiting barrier electrical resistance is a function of the type of material, its organic and/or inorganic content, thickness, density, hydrophilicity or hydrophobicity and porosity. The magnitude of barrier resistance can range from tens of ohms to millions of ohms (Meg Ohms). The diffusion limiting barrier on the working electrode limits the diffusion of species such as glucose and oxygen into the active zone to a fraction of their bulk concentration. The fraction of the bulk concentration can be, in some embodiments, between about 0% and about 90%, between about 0% and about 80%, between about 0% and about 60%, between about 0% and about 50%, between about 0% and about 25% or between about 10% and about 25%. If too much glucose diffuses into the active zone, there may not be sufficient oxygen, enzyme, or a fast enough rate of mediator turnover to yield a linear response over a wide dynamic range. For an analyte such as glucose, the diffusion controlled dynamic range can be 0-1000 mg/dL or 0-56 mmol/Liter (mM).

A potentiostat may be used to control the applied voltage or current to an electrochemical cell. FIG. 1 depicts (600) a portion of an electronic circuit for an exemplary potentiostat that can be used to apply a voltage or current between the working and counter electrodes of an electrochemical cell. The working electrode can serve as the positive or negative terminal of the electrochemical cell. The combination of the working and counter electrode can also constitute a conductivity cell. In addition, the potentiostat can be programmed to apply various voltage waveforms between the counter and working electrodes. In one embodiment a square wave voltage waveform is applied between the working and counter electrodes. The shape (e.g. square waveform) magnitude of the pulse (voltage) and the time period of its application, referred to as the pulse width (time in microsec to sec) can be controlled by the computer code resident within a computer operably connected to the potentiostat. The pulse can be applied at a constant or variable time known as the period (sec to minutes).

In some embodiments, a potentiostat FIG. 1, is used to interface with the sensor. The potentiostat can comprise a unique current-to-voltage converter circuit, allowing measurements to span a wide dynamic range using a low-cost 12-bit analog-to-digital converter (ADC). By the use of current mirrors, this circuit provides simultaneous analog outputs corresponding to several current ranges (e.g. 0-200 uA and 0-2000 nA). Current mirrors are typically constructed having a reference branch, through which the current to be mirrored is directed, and output branches, where a current equal to the reference branch current appears.

In some embodiments, the sensor's working electrode can be connected to a reference voltage supply (V_{ref}), such as from a bandgap voltage reference. This reference supply can also be used as a reference for a digital-to-analog converter (DAC) or voltage divider. The output of the DAC or voltage divider can be applied to the non-inverting input of an operational amplifier (OPA); the sensor's reference electrode is connected to the inverting input of an OPA. Generally, in some embodiments, an OPA with high input impedance and low leakage currents should be used to minimize the amount of current flowing in the reference electrode. In some embodiments, for proper circuit operation, current in the reference electrode should be less than one ten-thousandth of the current in the working electrode. If needed, a high-impedance buffer stage can be added between the reference electrode and the OPA. The output of the op amp can be connected via a switching element, such as a relay or a CMOS transmission gate, and via the reference branch of a first current mirror (made up of Q1 and Q2 in the diagram), to the sensor's counter electrode. The switching element, and the DAC if used, are controlled by software to create the desired pulsed waveform(s). For example, the software may open and close the switching element at predetermined times in order to create a square pulsed waveform.

Current from an output branch of the first current mirror can be connected to the reference branch of a second current mirror (in the diagram, made up of Q3, Q4, and Q5). This current mirror has one or more output branches, each directing its output current through a resistor (e.g. R1 or R2 in the diagram). The value of this resistor can be selected to convert a specific range of currents to a range of voltages by the application of Ohm's law. For example, to convert currents in the range of zero to 200 microamperes to voltages in the range from 0 to 2 volts, a 10,000 ohm resistor would be chosen. By construction of the current mirror with multiple output branches, several ranges of current can be converted to the same range of voltage. Thus, the circuit can achieve improved sensitivity to small currents while retaining the ability to measure larger currents.

In order to understand the response of an amperometric enzyme working electrode, both chemical and electrochemical reactions are considered. For example, in the absence of oxygen, glucose oxidase (its native form is oxidized) will oxidize glucose until all the enzyme is in its reduced form (equation 4), at which point, the reaction stops. If oxygen or another mediator is present, the enzyme can be reactivated so the catalytic cycle can continue until the entire supply of the mediator is oxidized (equation 5). If a continuous supply of oxygen or other mediator is present, the enzyme catalytic cycle will continue until all the glucose is irreversibly oxidized or the supply of analyte or substrate is depleted. As shown below, these reactions occur in the absence of an applied voltage.

Glucose+GOx (FAD⁺)→GOx (FADH₂)+Gluconic acid (chemical) (4)

GOₓ(FADH₂)+O₂ (Mₒₓ)→GOₓ(FAD⁺)+H₂O₂−(M_{red})−2e⁻ (chemical) (5)

H₂O₂+H₂O→HOO⁻+H₃O⁺pKₐ=11.6 base (chemical) (6)

Gluconic Acid (RCOOH)→Gluconate (COO⁻ X⁺) pKₐ=3.7 acid (chemical) (7)

HOO⁻+RCOOH→RCOO⁻+H₂O₂ (acid-base reaction) (8)

In equation 4 above, GOₓ represents glucose oxidase, the term (FAD⁺ or just FAD) represents the native, oxidized form of Flavin Adenine Dinucleotide, the active site within GOₓ responsible for electron transfer and, (FADH₂) represents the reduced form of FAD. There are two FAD groups within the GOₓ enzyme. In equation 5, Mₒₓ represents the oxidized form of a mediator and M_{red} represents the reduced form of a mediator and 2e⁻ represents the number of electrons transferred in the chemical reduction of oxygen to hydrogen peroxide (H₂O₂). Equation 6 represents the dissociation of hydrogen peroxide in the presence of water to produce the basic anion HOO⁻. In equation 7, the symbol X⁺ represents a cationic species such a proton (H⁺) or metal ion such as sodium (Na⁺) or potassium (K⁺). Gluconic acid is a weak acid in equilibrium with its anionic form as defined by the pKa of gluconic acid which is 3.7. In the absence of other effects, such as high pH, approximately 98% exists as gluconic acid and 2% exists as negatively charged gluconate.

Equation 8 shows that the anionic form of H₂O₂ may also serve as a base that de-protonates gluconic acid to its anionic form gluconate. Equations 4-8 serve to show that within the active zone of the working electrode, in the absence of an applied voltage, the conductivity within the active zone can vary due to chemical reactions. Referring to equation 5, by continually regenerating the oxidized mediator (Mₒₓ) from (M_{red}), the catalytic cycle can continue. For example, the reduced form of oxygen (Mₒₓ) is hydrogen peroxide (M_{red}). If there is a way to regenerate oxygen from hydrogen peroxide, the supply of dissolved oxygen can be replenished such that the concentration of oxygen in the bulk solution need not be present in high excess. The yield of oxygen from the electrochemical oxidation of hydrogen peroxide need not be 100%; however, enough may be regenerated to augment the supply of oxygen from the external bulk solution surrounding the working electrode, thereby reducing oxygen limitation at high glucose concentrations. Hydrogen peroxide can be oxidized to oxygen through the use of a platinum electrode in an electrochemical cell. In the presence of an aqueous electrolyte solution and a platinum working electrode poised at a potential that causes catalytic oxidation of hydrogen peroxide (e.g. +0.4 to +0.6 volts vs. Ag/AgCl), oxidation of hydrogen peroxide causes a current to flow, the magnitude of which is directly proportional to the concentration of hydrogen peroxide generated and the concentration of an analyte such as glucose.

If a platinum electrode is associated with or has bound to its surface an enzyme such as glucose oxidase; in the presence of glucose and dissolved oxygen, hydrogen peroxide will be generated in proportion to the mass of glucose oxidized.

Current generated from the electrochemical oxidation of hydrogen peroxide is directly proportional to the mass concentration (molality) of glucose consumed by the enzyme reaction. In general, this is a characteristic of amperometric enzyme working electrodes, where the concentration or activity of the analyte (e.g. glucose) is indirectly measured by the oxidation or reduction of a byproduct of the reaction of analyte with the enzyme (e.g. H₂O₂). This byproduct can be a reduced or oxidized mediator and, in the case of oxidase enzymes, the reduced mediator is either hydrogen peroxide and/or other mediator (Mᵣₑd). In the presence of an applied voltage the oxidized form of the mediator is regenerated so the oxidation-reduction cycle continues as shown in equations 9 and 10 below:

H₂O₂ (M_{red})→2H⁺+O₂ (Mₒₓ)+2e⁻(ne.⁻) (electrochemical oxidation) (9)

[M⁺²]_{red}→[M.⁺³]ₒₓ+1e⁻ (ne⁻) (electrochemical oxidation) (10)

Equation 10 is a simplified expression for the turnover of mediator. Mediators (Mₒₓ or M_{red}) are small molecules that can either oxidize or reduce the corresponding reduced or oxidized active site(s) within an enzyme (FAD/FADH₂) by shuttling electrons between the enzyme and an electrode surface.

Enzymes are typically large proteins having a three-dimensional structure. The active site of the enzyme may be buried within its three-dimensional structure and not subject to direct electrochemical oxidation or reduction because the distance of the enzyme active site from the electrode surface is greater than the distance associated with direct electron transfer (less than about 20 Angstroms). The oxidized or reduced form of the mediator is small enough to diffuse into the active site of the enzyme, accept or give up electrons, and return to the working electrode surface to be reduced or oxidized electrochemically; thereby, recycling the mediator to its active form. The electrode response generated, in the form of a current or voltage, from the electrochemical oxidation or reduction of the mediator, is directly proportional to the mediator concentration and the concentration of an enzyme specific substrate such as glucose. Mediators can include the oxidized or reduced form of metal ions such as Fe⁺³/Fe⁺² found in compounds such as ferri- and ferro-cyanides, organo-metallic compounds such as ferrocenes, polymer networks containing metals such as osmium, a quinone/hydroquinone couple or neutral molecules such as oxygen (O₂), the native mediator for glucose oxidase.

The electrochemical oxidation of hydrogen peroxide, shown in equation 9, produces 2 protons (2H⁺) that can result in a transient change in pH, within the active zone, and thus a transient increase or decrease in the conductance within the active zone.

Changes in the local pH or other ions may be temporary due to diffusion of hydrogen ions away from the electrode surface or the neutralization of hydrogen ions by buffer within the active zone, such as phosphate buffer. Whether the increase in conductance within the active zone is due to gluconate, gluconic acid, hydrogen peroxide anions or hydrogen ions, the slew rate of the op amp controlling the voltage to the counter electrode will increase when glucose is higher and decrease when glucose is lower.

### Pulsed Voltage Measurements

The voltage applied between a counter electrode and a working electrode is in some embodiments pulsed or applied intermittently using a waveform generator. For example, various kinds of waveforms (sine, triangular, square, etc.) can be applied between the working and counter electrodes. The waveform may be applied for a certain time period and then turned off.

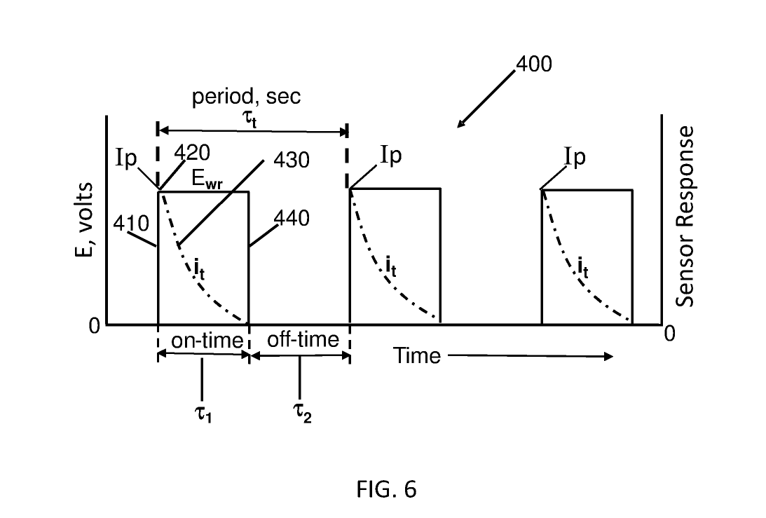

FIG. 6 shows a series (400) of intermittent square wave voltage pulses having a total period (τₜ), with a voltage on-time (τ₁) and a voltage off-time (τ₂);

where, τₜ=τ₁+τ₂ (11).

Referring to FIG. 6 and equation 11, if the total period τₜ is 5 seconds, the voltage on-time (τ₁) can be 0.3 seconds with an off-time (τ₂) of 4.7 seconds, or both the on-time and off-time can be 2.5 seconds, or any combination of (τ₁) and (τ₂) that adds up to the total period (τₜ). In FIG. 6, the application of a square wave voltage pulse, (410), gives rise to a current maximum Iₚ and a working electrode voltage maximum (420), which remains constant throughout τ₁. The voltage pulse also creates a working electrode response as a current vs. time transient, (430), with a maximum at (420). Under ideal conditions, within τ₁, the concentration of analyte at the electrode surface drops to near zero before the next pulse. Between pulses, the off-time period τ₂ allows the concentration of glucose oxidation products (equations 4-8) to increase so when another waveform pulse is applied there is another voltage vs. time response from the counter electrode and a new slew rate for the counter electrode response may be calculated. In addition, the working electrode peak response (Iₚ) or any current value beyond Iₚ, along the falling current transient (iₜ) (430), is directly proportional to glucose concentration. When the voltage is turned off, at the end of τ₁, the poise voltage, (440), falls to zero or the open circuit potential.

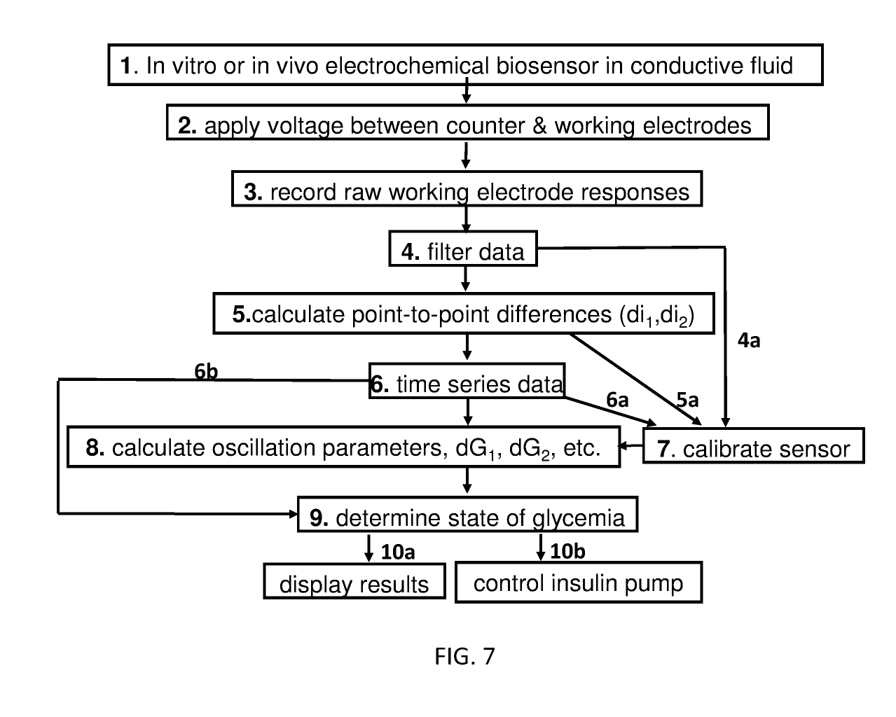

FIG. 7 shows a flow chart of how an embodiment of the present invention measures analyte concentrations or measures metabolic patterns in a liquid sample either in vivo or in vitro.

Step 1: Electrodes or electrochemical cell(s) are placed in a conductive medium (for example, dissolved electrolytes, buffer, water, subcutaneous body tissue, body fluid, etc.). Analytes may be already present within the conductive medium or analytes may be added to the conductive medium. A combination of electrodes or electrochemical cells may be in the form of (a) dry or wet strip onto which analyte samples are added, (b) combinations of electrodes suspended in a conductive medium or (c) electrochemical cell(s) that are placed completely or partially in vivo and surrounded by body fluid;

Step 2: Energy is applied between a counter electrode and working electrode of an electrochemical cell in the form of a voltage or current. The energy may or may not be pulsed at regular time intervals.

Step 3: The raw response of the counter electrode and/or working electrode of an electrochemical cell is recorded. One response may be the op amp voltage input to the counter electrode. A second response may be the output current response from a working electrode.

Step 4: The raw sensor response data from Step 3 may be filtered to smooth the data and remove high frequency noise. In step 4a, the filtered data may be used to calibrate the sensor response to analyte concentration.

Step 5: The filtered data from Step 4 may be used to calculate point-to-point differences in filtered sensor response versus time, thus creating a time series. If the filtered response is a current then first and second differences are calculated yielding di₁ and di₂ current responses versus time. Step 5 creates a time series of amplitude (di₁ or di₂) and frequency data (time). The point to point differences may also be used to calibrate the sensor as in step 5a. The di₁ or di₂ differences can be correlated with measured analyte concentrations to calibrate the di₁ or di₂ responses in terms of analyte concentration differences (dG₁, dG₂).

Step 6: is the un-calibrated time series created in step 5. For example, the di₁ or di₂ differences create a time series or metabolic profile or pattern that can be compared to similar time series data from subjects with different states of glycemia such a normal, impaired glucose tolerance, type 1 or type 2 diabetes to determine a given subjects state of glycemia. This information may be used directly to characterize state of glycemia without the necessity of knowing glucose concentrations. In step 6a, the time series data can be calibrated to glucose concentration; however, the pattern will look the same whether the differences are currents or glucose concentrations.

Step 7: The times series in step 6 and the calibration in step 6a can be used to obtain the metabolic profile in terms of analyte concentrations such as glucose or changes in glucose. In some cases, it may be advantageous to analyze a time series that has no zero or negative values. This can be accomplished by adding a sufficiently large constant glucose value (X) to all the dG₁ or dG₂ (point-to-point differences in glucose concentration) to obtain dG₁+X or dG₂+X values versus time such that all values will be positive, but the time series will maintain its overall structure with respect to the calculated differences. The two time series may be analyzed for characteristic time series such as amplitude (peak values) and frequency data (time difference between peaks), integrated peak area, average amplitude values and average frequency data to further characterize the metabolic profile to form a data base of such information from subjects having different glycemic states.

Step 8: The characteristic time series (step 6 or step 8) can be used to determine a subject's state of glycemia (Step 9). The data may be displayed to provide a graphical representation (10a) of the metabolic profile or bulk glucose concentration or data (10b) that may be used to control an infusion device such as an insulin pump. The bulk analyte concentration is the analyte level (e.g., glucose) prior to differencing. In addition, analyte concentrations calculated from the counter electrode response(s) may be averaged with corresponding analyte concentrations calculated from working electrode response(s) to yield a redundant, more accurate indication of analyte concentrations.

When prior art amperometric electrodes, such as amperometric enzyme electrodes used to measure analytes such as glucose or other analytes, are used, it is the response of the working electrode to changes in analyte concentration that is measured, and it is often a current response. For in vitro working electrode measurements, taken after the addition of an analyte, it is the steady state response of the working electrode that is measured and used to calculate analyte concentration.

Generally, the steady state response time of an in vitro working electrode is defined as the time to reach an equilibrium state where the signal is flat or where the average point-to-point difference is not more than 10%. This response time is measured in minutes.

In some embodiments, provided is a method of analyzing cellular metabolic oscillations of a subject comprising inserting a biosensor within dermis of the subject, applying energy to the biosensor, recording and storing raw output response data from the biosensor for a period of time, filtering the raw output response data to provide filtered response data, calibrating the filtered response data versus an oscillating cellular substance versus time utilizing the first or second response differences and a concentration of a reference sample from a fingerstick or venous blood draw wherein the concentration of the substance is determined using an in vitro method of measurement, storing the calibrated filtered response data versus time, detrending the calibrated filtered data by taking a first or second order difference to obtain a series of peaks and valleys versus time, and measuring the time period between consecutive peaks and the amplitude of the peaks.

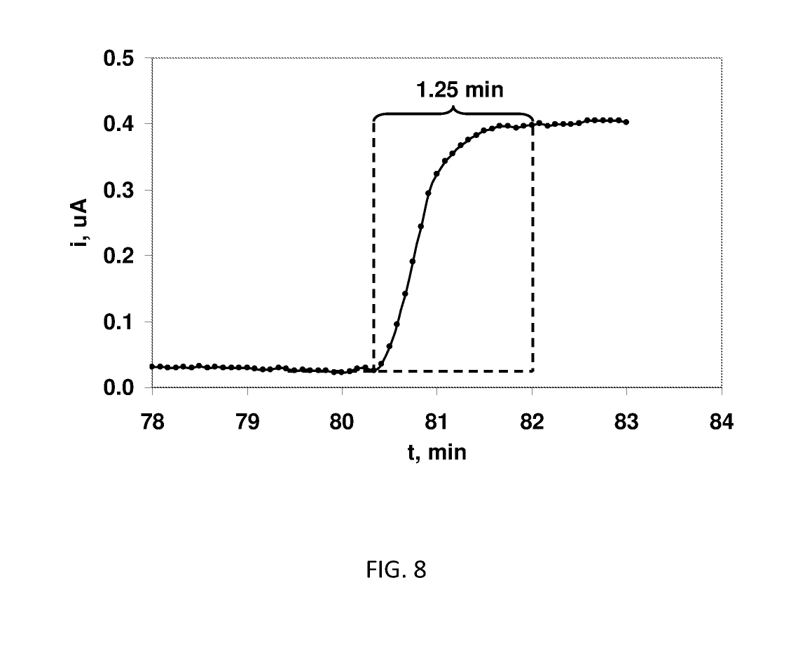

An example of the in vitro measurement of a working electrode response time of an amperometric enzyme electrode in pH 7.4 PBS is shown in FIG. 8 wherein the time to reach a steady state response is estimated to be 1.25 min.

As compared with in vitro response times, in vivo response times are generally expected to be comparatively longer and more difficult to measure. The in vivo measurement of working electrode response time is difficult to determine due to the lack of a steady-state equilibrium. Dynamic changes occurring in tissue fluid adjacent to the implanted working electrode, physiological lag time associated with glucose transport across the endothelium of capillaries into interstitial fluid, oscillatory cellular consumption of the analyte can contribute to a non-steady state response. In vivo, the concentration of substrates or analytes, such as glucose, may be at a steady state for only brief periods of time. However, not knowing contributions from other processes, parsing the response time of the electrode from other in vivo dynamic processes may require some adjusting of operational parameters.

As opposed to continuous in vivo measurements, continuous in vitro measurements of analyte concentration can be observed in discrete steps because there are only very minute changes in the bulk concentration of the analyte due to working electrode consumption. There are no competing physiological reactions consuming the analyte as is the case in vivo. When a diffusion limiting barrier covers the working electrode and there is adequate diffusion control, in vitro electrode response is not affected by mixing or stirring at the outside surface of the diffusion barrier. As a result, discrete stepped responses are easily distinguished and may be used to determine sensor response time.

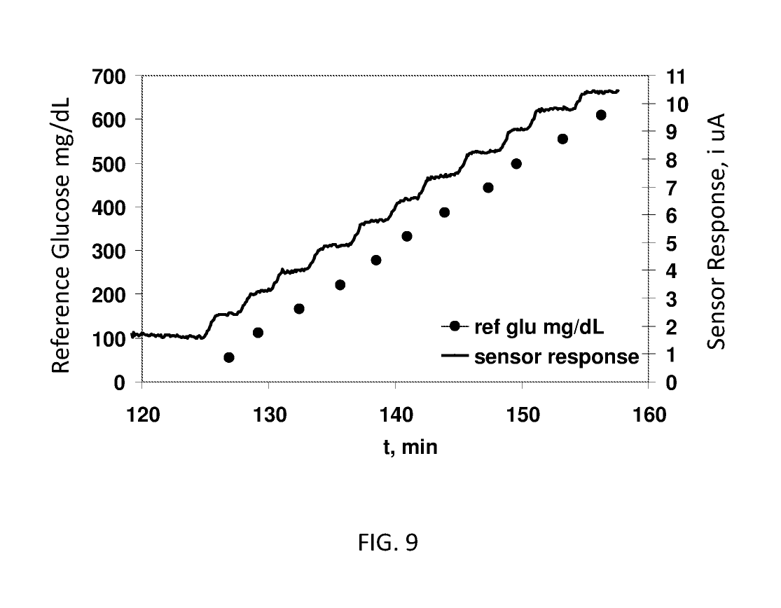

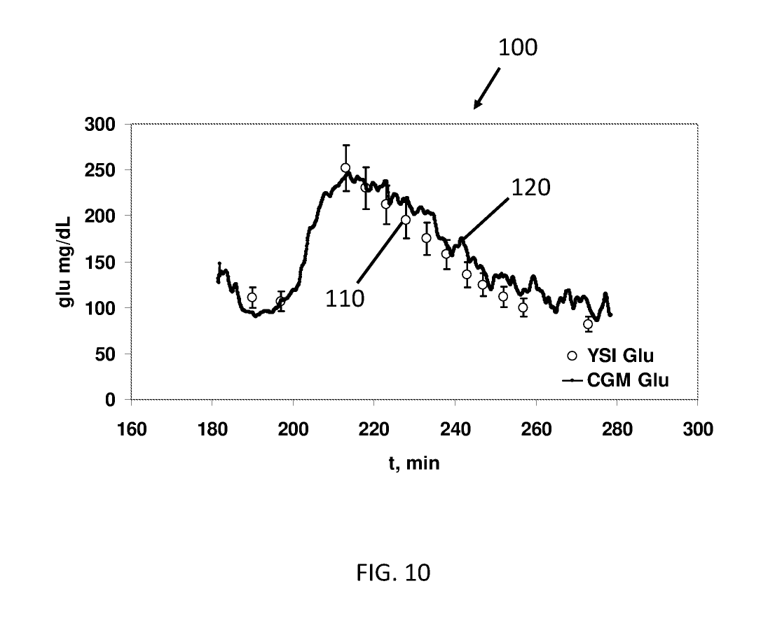

An example of this type of response, from increasing glucose concentrations, is shown in FIG. 9. This is in contrast to continuous in vivo measurements shown in FIG. 10; wherein, the working electrode response from a continuous glucose sensor exhibits a dynamic continuum with no discrete steps. Under these conditions, measurement of sensor response time, in and of itself, is confounded by uptake of glucose by cells and the physiological lag. The current response from a working electrodes takes longer (minutes) than the time for the voltage at the counter electrode to settle (microseconds).

The measurement of dynamic changes in the voltage output to a counter electrode can be accomplished in microseconds such that the in vitro and in vivo response time of a sensor is essentially eliminated, which reduces the overall lag time of an in vivo sensor. For example, the total lag time of in vivo glucose sensors can be on the order of about 20 minutes which includes both physiological and sensor response time. Reducing the sensor response time, reduces the total lag time such that more accurate real-time measurements may be made.

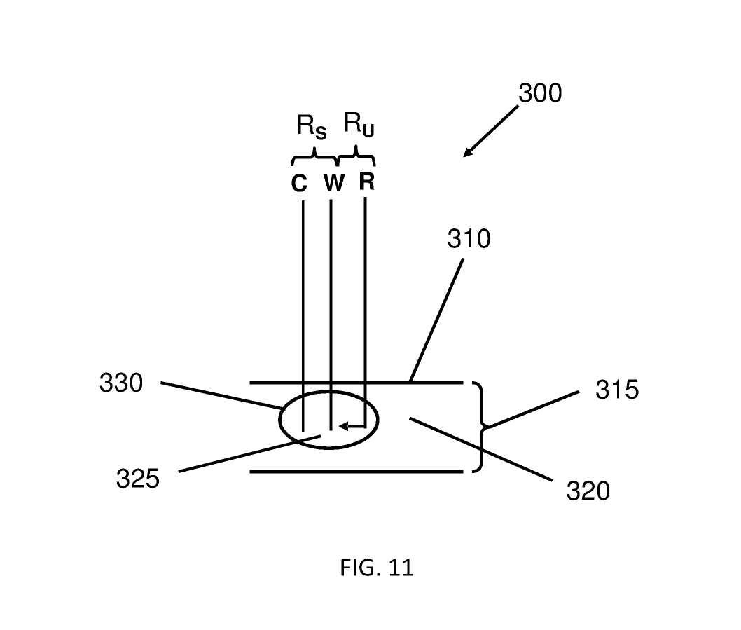

FIG. 11 shows an example of an in vivo configuration for an implanted three-electrode cell (300) including an amperometric enzyme working electrode (W), a counter electrode (C), a reference electrode (R), a skin surface (310), a skin thickness (315), subcutaneous tissue and interstitial fluid (ISF) (320), an active zone (325), a diffusion limiting barrier (330), resistance (Rₛ) between the working and counter electrodes and uncompensated resistance (Rᵤ) between the working and reference electrodes.

In FIG. 11 all three electrodes are shown implanted within subcutaneous tissue (320) and are encapsulated within a diffusion limiting barrier (330). The application of a diffusion limiting barrier over the electrodes leaves a small space or interface (325) called the active zone, between the inside surface of the diffusion limiting barrier and the working or counter electrode, that serves as a path of fluid communication between the implanted electrodes and surrounding body fluid. This cell geometry is near the ideal configuration for reducing R_{S}. In the ideal electrochemical cell, the counter and working electrodes are as close together as possible to minimize R_{S}, and the reference electrode is as close as possible to the working electrode, without shielding the working electrode surface, to minimize Rᵤ, the uncompensated resistance between the working electrode and reference electrode. Even with ideal cell geometry, the act of implantation of a three-electrode electrochemical cell may still elicit an inflammatory or biofouling response to the implanted sensor(s).

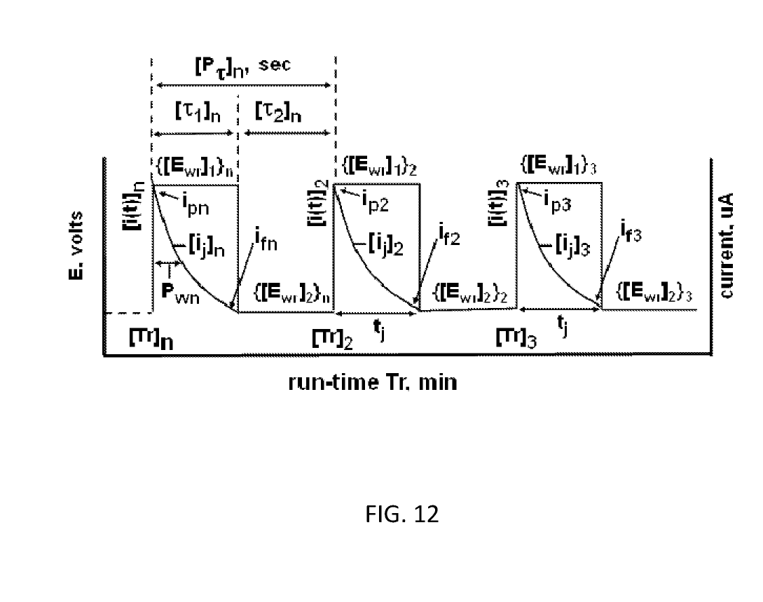

In FIG. 12, in response to each voltage pulse {[E_{wr}]₁}ₙ, each sensor current transient [ij]ₙ rises steeply to a peak value, represented by the symbol [iₚ]ₙ; after which, it declines exponentially to a final current value i_{f}n at the end of the pulse width period. The subscript n (n=1, 2, 3 . . . ) indicates each current transient is indexed to a discrete value of the run-time [Tr]ₙ. Each run-time point [Tr]ₙ is defined as the time when the voltage pulse begins, the subscript j (j=1, 2, 3 . . . ) represents declining transient currents [iⱼ]ₙ and corresponding transient times tⱼ after the peak current and the maximum value of subscript j is a function of the sampling rate (Hz) and the pulse width period (τ₁). For a diffusion controlled process, the post peak transient current, is defined by the Cottrell Equation:

i=nFACⱼ^{o}Do^{1/2}/(πt)^{1/2} (14)

where,
i=the sensor current on the falling portion of the current transient in Amp
n=number of electrons transferred, equivalents/mol (1, 2, 3 . . . )
F=Faraday constant, 96,485 Coulombs/equivalent
A=electrode area, cm²
Cⱼ^{o}=initial mass concentration of the analyte, mol/cm³ (molality)
Dⱼ=initial diffusion coefficient of the analyte, cm²/sec
t=transient time, sec.

The transient current is inversely proportional to the square root of transient time tⱼ; and, for a diffusion-controlled reaction at a planar electrode, the product i*(tⱼ^{1/2}) should be constant. In addition, there is a linear portion of the exponentially declining current transient that begins at the peak current i₁ and ends at a time tⱼ where the current becomes non-linear. This linear region exists for approximately 2-100 msec after the peak current.

Sensor currents referred to herein may consist of discrete single transient currents [iⱼ]ₙ, the difference between two transient currents [i₂−i₁]ₙ, an average transient current, the rate of change of the transient current or integrated transient current expressed as charge in coulombs, in accordance with Faraday's Laws where charge is expressed as a change in current multiplied by a corresponding change in time.

In order to obtain calibrated values of an analyte concentration, each discretely sampled indexed transient current [iⱼ]ₙ, integrated transient current or function of the transient current used as a sensor output response, for the calculation of an analyte concentration, must be calibrated against known analyte concentrations so that calibration parameters such as sensitivity and intercept may be determined.

In FIG. 12, at each voltage pulse beginning at [Tr]ₙ, (n=1, 2, 3, . . . ), the voltage rises from a baseline magnitude of [E_{wr}]₀ to the maximum of the poise potential [Ewr]₁. The magnitude of [Ewr]₁, is preferably selected to enable an optimized rate of an electrochemical redox reaction. The maximum may or may not be the diffusion limited rate. After a time period defined by the pulse-width τ₁, [Ewr]₁ may be stepped to [Ewr]₂ for the duration of the inter-pulse period T₂. The magnitude of [Ewr]₂ is preferably chosen such that the electrochemical redox reaction (e.g. electro-oxidation of H₂O₂) still proceeds, but at a reduced rate versus the rate at [Ewr]₁. When [Ewr]₂ is less than [Ewr]₁, the concentration of the analyte species within [Ewr]₂ will be greater than its concentration within the pulse width period, τ₁, of [Ewr]₁. With respect to amperometric glucose oxidase biosensors, the oxidation of glucose by GOₓ proceeds in the absence of an applied potential such that hydrogen peroxide may increase during the inter-pulse period.

### Glucose Processing System

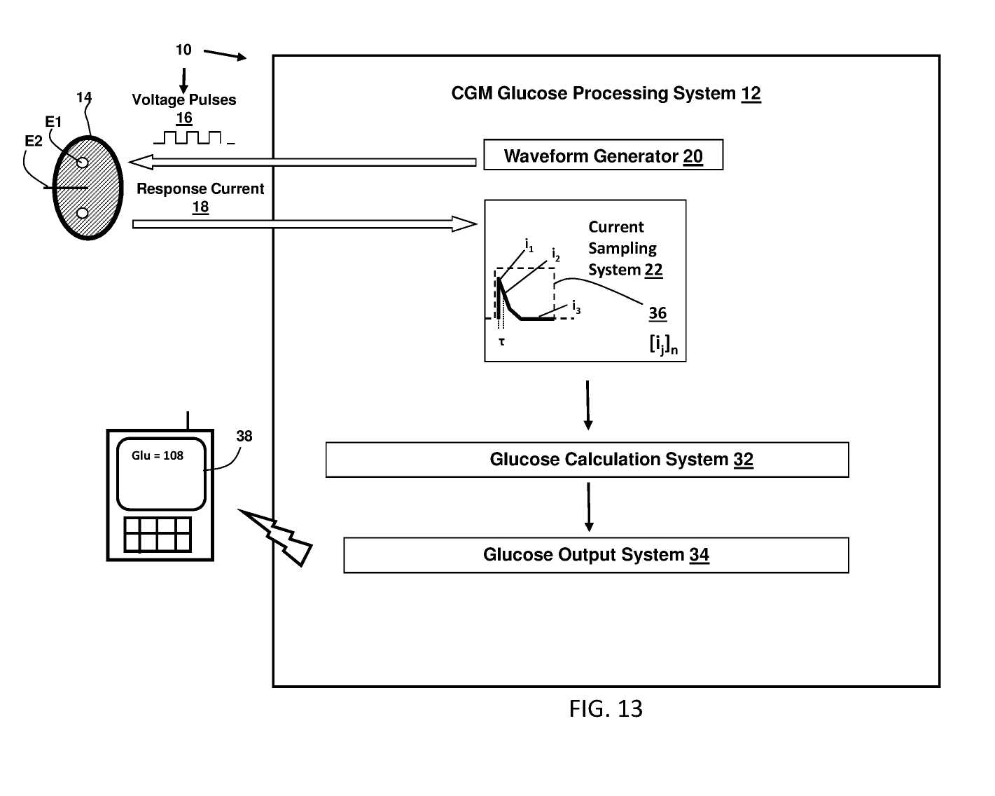

Referring now to FIG. 13, a system, 10, for capturing continuous glucose readings. The readings from sensor 14 are used by a Glucose Processing System 12 to calculate glucose readings. The applied voltage may consist of a steady state voltage or voltage pulses, 16, at any frequency, and comprise any shape (e.g., a square wave, triangle, etc). The Glucose Processing System, 12, includes: a potentiostat incorporating a Waveform Generator 20 for generating and applying steady state, periodic or non-periodic voltage waveforms to the biosensor; a current sampling system, 22, for sampling the response current, 18, from application of the voltage waveforms to produce sampled currents, 36; a Glucose Calculation System, 32, for calculating a glucose reading from sampled currents 36; and a Glucose Output System 34 for outputting the glucose reading to the display device 38. Glucose Processing System 12 can calculate a glucose reading using currents generated from the application of any applied voltage waveform 16 (square waveform shown) as often as desirable. Moreover, some or all of Glucose Processing System 12 may be integrated with the sensor 14 or reside apart from the sensor 14 (e.g., within display 38).

Once the glucose concentration is calculated, it can be sent by Glucose Output System 34 to an output device 38. Output device 38 may comprise any device capable of receiving and displaying data (e.g., an insulin pump, a smartphone, a Bluetooth enabled device, a watch, etc.).

(a) The biosensor housing, 14, containing the biosensor working electrode and at least one other electrode is attached to the skin of a subject using an adhesive pad on the underside of the housing. The liner over the pad is removed and the biosensor housing pressed against the skin.
(c) The biosensor within the biosensor housing is activated by insertion into the subject, at which time, a potentiostat is triggered to begin an applied voltage regime.
(d) The applied voltage regime may consist of the application of a steady-state voltage or a series of periodic voltage waveforms, such as a square wave voltage pulse between a counter and working electrode. The initial potential, prior to the first voltage application, may be zero volts with respect to the reference electrode; greater or less than zero volts with respect to the reference electrode; or, an open circuit potential, E_{oc}. The steady state current response or the entire current transient generated from the application of the square-wave voltage pulses or a series of sampled transient currents are stored in the memory of the in vivo biosensor's microprocessor controlled monitoring unit, 38.
(e) A period is required for the in vivo biosensor to equilibrate to its surroundings. An example of such an equilibrium period is 60-120 minutes from the time of implantation. Throughout the run-time period, T_{R}, each application of a voltage creates a characteristic steady state current response or by voltage pulsing, a current transient, 36. Within each current transient, there are j values of current, [iⱼ]ₙ, after the peak current iₚ. The maximum value of j is determined by the pulse width and the data sampling rate.

In many endocrine systems, oscillations in the output of hormonal cells result in pulsatile release rather than continuous excretion. Insulin is a hormone that is essential for glucose metabolism and, in normal individuals, is released in pulses from the pancreas. Insulin pulse profiles from the pancreas consist of a non-pulsatile basal rate with superimposed, periodic secretory bursts. Although islet cells of the pancreas may exhibit rapid oscillations of insulin release, they may not always be observed in peripheral blood circulation. What is usually seen is a damped, integrated, rhythmic pattern.

Within the blood, the entrainment of high frequency insulin pulses with glucose oscillations is regarded as a sign of normal pancreatic function. Impairment of pulsatile insulin secretion or the lack of entrainment of high frequency insulin oscillations with glucose oscillations are believed to be early signs of β-cell dysfunction. In cases of impaired glucose tolerance, even though the frequency of insulin oscillations may be similar to normal individuals, the amplitude of the pulses may be decreased. These early signs of diabetes are believed to be contributing factors in the development of impaired glucose tolerance and insulin resistance.

In contrast to measurements in blood that require drawing blood from a subject, various embodiments described herein provide a way to measure glucose oscillations at the cellular level in interstitial fluid as a more convenient way of measuring and characterizing aberrations in glucose metabolism without the necessity of hospitalization or drawing of frequent venous blood samples. The examples listed below show that there are differences in cellular glucose oscillations in normal subjects versus those with type 1 or type 2 diabetes. In people with type 1 diabetes, insulin/glucose oscillations in blood are non-existent; however, they may still be present at the cellular level.

In some embodiments described herein, the measurement of glucose oscillations in interstitial fluid is used as a diagnostic test for abnormal glucose metabolism. Glucose oscillations in interstitial fluid have not previously been observed or used in a diagnostic setting. In order for glucose to reach cells it must diffuse across blood vessels and into the interstitial fluid where it can be transported to the cells to be metabolized to produce energy. Cellular glucose metabolism is known to oscillate, but real-time, in vivo measurements of cellular glucose metabolism have not been previously used for diagnostic purpose or to control insulin dosing. In some embodiments described herein real-time in-vivo measurements of cellular glucose metabolism are used to assess glucose metabolism and diagnosis disease states and potential disease states.

### In Vivo Metabolic Fingerprint

The term metabolomics may be defined as the systematic study of the unique chemical fingerprints that specific cellular processes generate (Daviss, Bennett. “Growing pains for metabolomics”. The Scientist, 2005; 19(8):25-28)′. Metabolomics is generally the study of small molecules with molecular weights less than about 1000 Daltons which are intermediates or products of cellular metabolism that give rise to characteristic metabolic profiles. The term metabolome generally refers to the total collection of intermediates and products utilized and produced in biological cells, tissues, organs and organisms. In combination, the intermediates and products of metabolism may be defined as analytes. There are numerous techniques for generating metabolic fingerprints. Early on, researchers analyzed and classified urine by odor, taste or the response by insects such as ants to specific metabolites indicative of certain disease states such as diabetes. Current analytical techniques include capillary electrophoresis, infrared spectroscopy, Raman spectroscopy, gas and liquid chromatography, mass spectrometry, nuclear magnetic resonance and combinations of these techniques. None of these analysis techniques can be used directly within the body. They require methods to remove samples from living tissues or cells followed by ex vivo analysis; however, urine collected external to the body can be analyzed directly.

In contrast to the above methods, and in the context of the present embodiments disclosed herein, in vivo metabolic fingerprinting refers to the time varying changes in the concentration of specific intermediates or products of metabolism such as glucose measured with a specific sensor directly in contact with living tissue or cells. Although the metabolic fingerprint for glucose may be different for each individual; there are features of the profile that can be related to aberrations in glucose metabolism which are revealed when comparing in vivo metabolic profiles from people with normal glycemia to those with impaired glucose tolerance, type 1 or type 2 diabetes. Using a plurality of in vivo analyte specific sensors it is possible to simultaneously obtain a plurality of metabolic fingerprints for different individual analytes. In the case of glucose sensors, they can be implanted within various layers of the skin such as the dermis or adipose tissue or they may be implanted in organs such as the brain, heart, kidney or liver. Sensors can also be implanted within blood vessels.

For example, to obtain an in vivo glucose metabolic profile, a minimally invasive, continuous glucose sensor is implanted within the skin such that the sensor or a cannula containing the sensor is inserted through the skin to a depth (e.g., about 3-5 mm) consistent with the measurement of glucose within interstitial fluid (ISF) surrounding and in contact with cells within the skin. A minimally invasive amperometric glucose oxidase sensor comprised of either two or three electrodes (anode-positive terminal has glucose oxidase on surface, cathode-negative terminal and optionally a reference electrode) is implanted transdermally and is operably connected to an on skin receiver and display that applies a voltage, using a potentiostat, between the positive electrode (e.g., glucose oxidase sensor) and the negative electrode (e.g. platinum wire) the receiver records the sensor response as current versus time and can display the sensor output as current or if calibrated, as glucose concentration versus time.

When glucose within ISF, that is surrounding cells, reaches the glucose oxidase on the positive electrode it is oxidized to produce gluconic acid and hydrogen peroxide. The positive voltage applied to the anode causes the oxidation of hydrogen peroxide which results in current flow. The magnitude of the current is directly proportional to the concentration of glucose according to the linear equation y=mx+b, where y is the current response (nano amps or micro amps), m is the slope of the line of current response (Y-axis) vs. glucose concentration (X-axis) in units of current per unit of glucose concentration (e.g., nanoamps/mg/dL or nanoamps/mmol/L), x is the glucose concentration and b is the background current in the absence of glucose. There are several ways of calibrating the in vivo sensor. The current response of the sensor can be related to glucose concentration by using factory determined slope and intercept or by drawing blood from a subject (venous or capillary blood), measuring the glucose and inputting the measured glucose concentration into the receiver. This calibration process can be performed over a time period that encompasses a range of glucose concentrations such that linear regression may be used to determine the slope and intercept.

Because the in vivo sensor is in contact with cells, cellular glucose oscillations will be included within the total sensor response (bulk concentration) to the glucose within the ISF as illustrated in FIG. 21. Due to increased sensitivity due to voltage pulsing of the biosensor, the sensor can measure the oscillating flux of glucose into the cell which will be superimposed on the overall signal for glucose concentration within the ISF. In the absence of knowledge regarding cellular glucose metabolism, the small oscillations in the flux of glucose into cells might be interpreted as “biological noise”. However, if cellular glucose oscillations can be extracted from the total glucose sensor response, these oscillations may be observed and characterized.

To extract cellular metabolic oscillations, to obtain a metabolic fingerprint, the point-to-point difference is taken between consecutive sensor output data points versus time. This produces either a plot of difference in glucose (dG₁) or difference in current (di₁) versus time. To remove positive or negative trends in the data, called detrending, a second difference between consecutive dG₁ or di₁ points versus time may be taken to produce a plot of dG₂ or die versus time. This process produces a time series or metabolic fingerprint in an oscillatory pattern consisting of peaks and valleys corresponding to changes in glucose concentration at the sensor-cellular interface that may be filtered and further analyzed. For example, measuring the peak amplitudes, area under the peaks and the time period between peaks or frequency would be one way of characterizing the metabolic fingerprint. Various statistical analyses such as mean amplitude, mean area under the peaks, mean frequency may be performed on the data to further characterize the metabolic fingerprint and provide a means of “scoring” a given metabolic fingerprint.

The examples described below provide evidence to support our observations that aberrations in cellular glucose metabolism within interstitial fluid, caused by diabetes, exhibit as irregular changes in cellular glucose metabolism, regardless of the stage of disease whether type 1 or type 2 diabetes, that can be quantified to determine the extent of metabolic disease. Though not wishing to be bound by theory or analogy, the significance of this invention, in various embodiments disclosed herein, can be analogized to a combustion engine in an automobile. Measuring glucose in blood provides information on how much fuel is in the tank, but what one really needs to know is how efficiently the fuel is being burned by the engine, in this case metabolized by cells. The blood stream transports glucose to the cells. Glucose in blood crosses the walls of the capillaries into the interstitial fluid (ISF or lymph) where it is transported to the cells. If the process of cellular glucose metabolism and/or insulin utilization is impaired, glucose in ISF builds up beyond normal levels leading to altered cellular glucose metabolism and the complications of diabetes.

An algorithm or mathematical method can be used to process data from cellular glucose metabolism within interstitial fluid. Using a set of rules or scoring criteria, the measured metabolic fingerprint, for example, composed of frequency and amplitude data, is examined for defects by comparison to a database containing metabolic fingerprints of cellular glucose metabolism under normal and abnormal physiological conditions. If the measured metabolic fingerprint is outside the range or limits of normal metabolic fingerprints, the degree to which the measured metabolic fingerprint is abnormal may indicate the early onset of a metabolic disease or the extent of a metabolic disease. This invention is particularly useful for diagnosing the early signs of diabetes. In addition, analysis of the frequency and/or amplitude of the metabolic oscillations may be used to calibrate an in vivo sensor or control insulin infusion from an insulin pump.

Disclosed herein, in some embodiments, is a convenient method for measuring subcutaneous glucose fluctuations within the body as a means of obtaining metabolic information indicative of the state of glycemia. The information so obtained can be used for diagnostic purposes, to derive a metabolic profile of metabolism, to predict the future onset of disease, to provide a user calibration-free means of measuring glucose or to provide a user calibration-free closed loop means of administering a drug or hormone.

Disclosed herein in some embodiments, are systems, methods, and devices for in vivo analysis, in real-time, of temporal fluctuations in the level of biological constituents within mammalian body fluids such as interstitial fluid for the purpose of diagnosing or treating diseases.

Described herein, in some embodiments, is a new in vitro model of cellular glycolysis that can be used for characterization of glycolytic oscillations in yeast or other types of cells including cancer and stem cells. The methods developed for analysis of the in vitro model can be used to characterize in vivo metabolic oscillations within the ISF of mammals. In various embodiments, this will enable the development of new products for screening individuals for early signs of abnormal glucose metabolism and give researchers new tools for unraveling how cellular glycolysis and other glycolytic oscillations are affected by insulin resistance or other disease states such as cancer. Products developed from the methods, systems, and devices of the disclosed invention, in various embodiments, will enable more accurate measurements with continuous glucose monitors (CGMs) and more precise and early screening of individuals at risk for diabetes. Application of various embodiments described herein will result in a closed loop insulin delivery system that relies on information from cellular glucose metabolism thus providing a reliable bio-mimetic artificial pancreas.

In some embodiments described herein, is a device such as a biosensor that can be implanted within the skin and used to track metabolic fluctuations in cellular metabolism within a living body. The biosensor can be an electrochemical biosensor, such that it forms part of an electrical circuit or electrochemical cell. For example, a biosensor can be polarized with a voltage sufficient to oxidize or reduce a chemical species such as glucose. The signal produced by the biosensor can be directly or indirectly proportional to the concentration of chemical species found within body fluid. Fluctuations in the level of the biosensor signal output are reflective of fluctuations in the concentration of molecules within body fluid at the cellular interface. Such fluctuations in concentration may result from the uptake of metabolic intermediates by cells or the production of products of cellular metabolism.

In another aspect of embodiments described herein, a mathematical algorithm can be used to extract times series data in order to analyze metabolic processes occurring within interstitial fluid. The results obtained from the mathematical algorithm are a pattern of metabolic fluctuations or oscillations. For example, in a human with a normal functioning pancreas, the period between metabolic oscillations may be regular within a defined time period with constant amplitude. The regularity in the pattern of glucose metabolism in a mammal, with a normal functioning pancreas, remains so despite changes in blood glucose levels. For mammals with a normal functioning pancreas, blood glucose levels remain within a fairly well defined range of approximately 80-140 mg/dL.

In a mammal with a malfunctioning pancreas, insulin release may be abnormal resulting in irregular glucose metabolism with episodic rather than a regular pattern of glucose metabolism. Glucose levels may rise above the normal range and may routinely exceed 200 mg/dL, resulting in a condition known as hyperglycemia. Conversely, glucose levels may rapidly fall below the normal range (˜80 mg/dL) and result in a dangerous condition known as hypoglycemia. The abnormal rise and fall, i.e., higher than approximately 180 mg/dL and lower than approximately 80 mg/dL, gives rise to irregular patterns of glucose metabolism as manifested in the amplitude of the oscillations. In some embodiments, described herein, irregular patterns of glucose metabolism can be used for diagnostic purposes. In some embodiments described herein, detecting irregular patterns of glucose metabolism can be used to indicate early onset of diabetes.

In some embodiments, a method of utilizing cellular oscillation data comprising inserting a biosensor within the dermis, applying energy to the biosensor, recording and storing raw output responses of the biosensor, filtering raw data, calibrating filtered response data versus time, detrending calibrated filtered data by taking a first or second order difference to obtain a series of peaks and valleys versus time representing, for example, point to point delta glucose, (dG₁, dG₁+X, dG₂, dG₂+X, di₁, di₁+X, die or di₂+X vs. time), using time series analysis to extract amplitude and frequency data, measuring the time period between consecutive peaks (frequency) and calculating the average period, standard deviation (SD), and area under the peaks; determining average area and SD, determining mean and standard deviation of glucose corresponding to peaks.

### EXAMPLES

### Example 1

Preparation of Glucose Oxidase Sensors: 80% Pt-20% Ir wires (0.014″ dia) were dip coated in a 3% solution of glucose oxidase in 5% BSA in pH 7.4 phosphate buffered saline. The sensors were dried in a mechanical convection oven at 50-55° C. for 15 minutes. This was followed by crosslinking by dipping the sensor into 5% glutaraldehyde in 0.1M bicarbonate followed by oven drying at 50-55° C. The dried sensors were dipped into a 3% solution of polyurethane (Tecothane-Lubrizol) dissolved in tetrahydrofuran (THF). The sensors were oven dried at 50-55° C. for 30 minutes. The sensors were tested for response to glucose in pH 7.4 phosphate buffered saline (PBS) using a three-electrode electrochemical cell and a potentiostat. Glucose response data from the potentiostat was transmitted wirelessly to a personal computer (PC) for storage and analysis. Typical stepped, diffusion controlled responses to increasing glucose concentrations were observed (as in FIG. 9).

Immobilization of Intact Yeast Cells on Glucose Sensors: A suspension of Saccharomyces Cerevisiae (Sigma, cat no. YSC2) was prepared by adding 50 mg of yeast granules to 1 mL of 0.05 M pH 6.8 phosphate buffer containing 1.0 g/L KCl, 10 g/L NH₄SO₄, 0.5 g/L MgSO₄-7H₂O, 0.5 g/L peptone and 3 g/L yeast extract in a 2.0 mL centrifuge tube. The suspension was gently rocked for 30 minutes at room temperature. The polyurethane coated glucose sensors from above, were dip coated with a solution of 0.5% hydrophilic polyurethanes (e.g. Techophilic-Lubrizol) dissolved in THF and cured for 30 min at 50° C. The glucose sensors were then dipped into a gently stirred suspension of yeast cells and cured for 30 min at 35° C. Once the yeast cells were dried onto the surface of the glucose sensor, they were anchored in place by dipping the sensors once again into the hydrophilic polymer mixture from above followed by oven curing for 30 min at 35° C.

Activity of Immobilized Yeast Cells: Experiments were carried out at a constant glucose concentration (200-250 mg/dL) in a three-electrode cell open to the air so that oxygen could freely diffuse into the buffer within the electrochemical cell. The yeast cell coated glucose oxidase sensor acted as the working electrode or anode, a coil of platinum wire as the counter electrode or cathode and a silver-silver chloride wire as the reference electrode. Oxygen is used because under anaerobic conditions, the glucose oxidase sensor would not respond or it would show a diminished response or saturate at low glucose concentration. A glass cylindrical electrochemical cell measuring 1.13″ high by 0.875″ in diameter was filled with 5 mL of the same growth medium used to prepare the yeast suspension above. The solution was stirred magnetically using a flea size stir bar at 50 rpm. A steady state voltage of +0.5 v (vs. Ag/AgCl) was applied to the glucose sensor using a potentiostat. The glucose concentration was adjusted to 220 mg/dL by the addition of microliter amounts of 50% glucose dissolved in PBS.

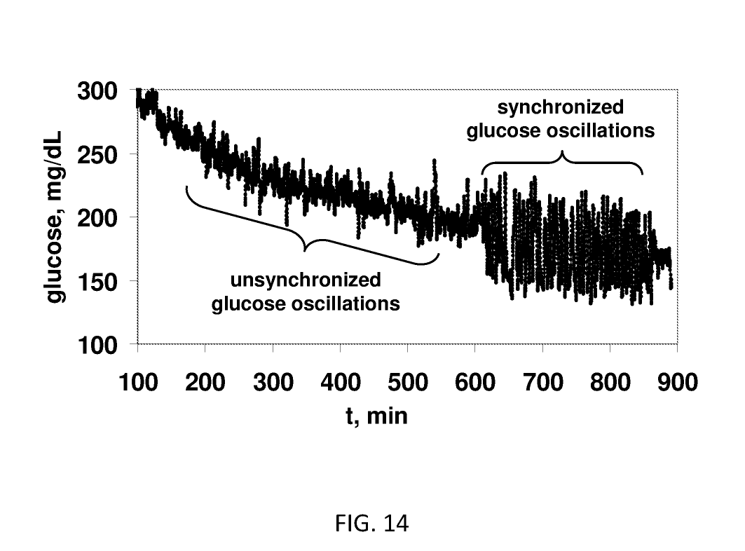

The level of glucose within the test cell was confirmed by testing on a Yellow Springs Instruments Model 2300 Glucose Analyzer. Glucose response data was transmitted wirelessly to a PC for storage and analysis. FIG. 14 shows the results of an experiment using a glucose sensor with yeast cells immobilized on the surface. This is an example of how the cells can take time to develop sustained, synchronized glycolytic oscillations. The negative drift in the sensor response is indicative of the system reaching an equilibrium state. The data within the first 600 minutes was chaotic and unsynchronized. As shown FIG. 14, it took about 600 minutes for the oscillations to become synchronized.

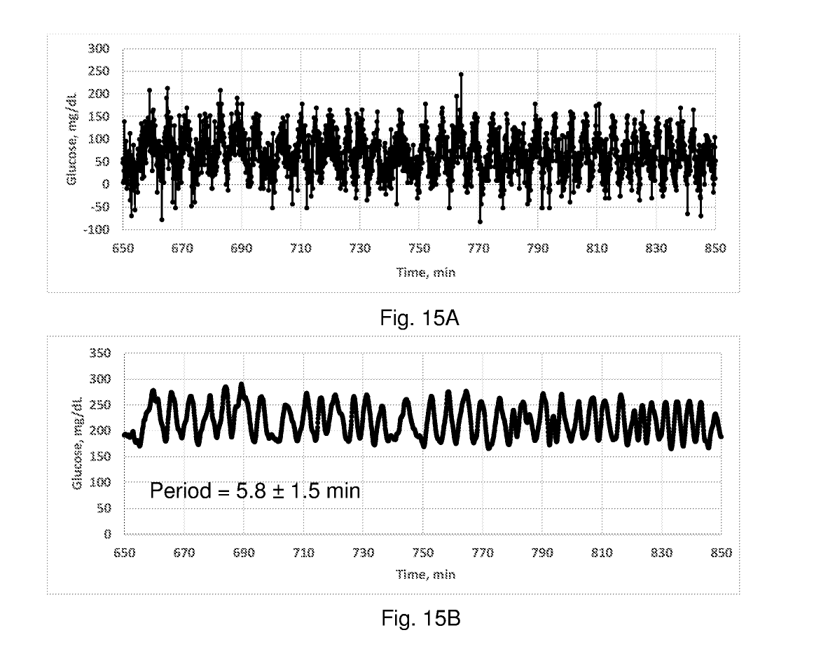

FIGS. 15 A and 15 B are an expanded view of the data between about 600 and 850 min in FIG. 14. FIG. 15A is the raw, unfiltered data showing synchronized glucose oscillations from clusters of yeast cells at different levels within the hydrophilic membrane. They have slightly different amplitudes depending on the amount of bulk glucose (G_{b}) available within the layers of cells. FIG. 15B is the low pass filtered data from FIG. 15A.

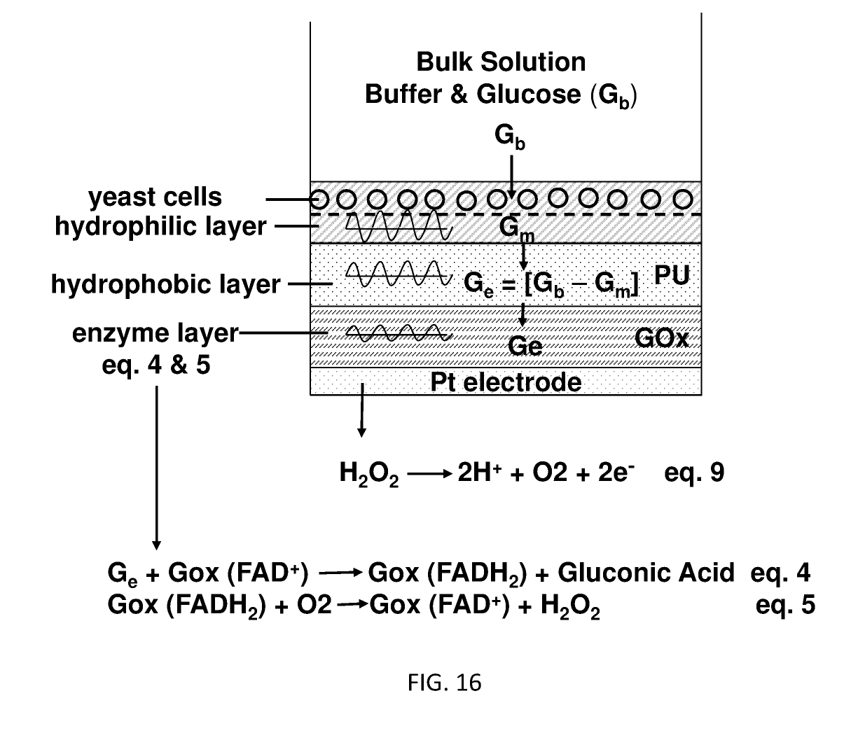

Measurement of glucose oscillations in Yeast cells: Referring to FIG. 16, assuming the immobilized yeast cells are intact, glucose is transported across the outer hydrophilic membrane into the layer of yeast cells where it is metabolized. As shown in FIG. 16, what the glucose sensor is measuring is the remaining, extracellular glucose (Gₑ), which is the difference between the bulk glucose (G_{b}) that diffuses into the hydrophilic polymer/yeast layer from the bulk solution minus that which is metabolized by the cells (Gₘ). The oscillating extracellular glucose (Gₑ) diffuses across the hydrophobic polyurethane membrane into the enzyme layer having glucose oxidase where it is oxidized by glucose oxidase to hydrogen peroxide and gluconic acid (equations 4 & 5). The hydrogen peroxide is electrochemically oxidized at the Pt (platinum) electrode to produce oxygen, protons and electrons, as in equation 5. The resulting current produced by the electrochemical oxidation of hydrogen peroxide is not only proportional to Gₑ, but also proportional to the glucose concentration (G_{b}) within the bulk of solution outside the layers over the platinum electrode. The protons produced in the electrochemical oxidation of hydrogen peroxide in equation 5 are neutralized by the external buffer solution which bathes the enzyme layer.

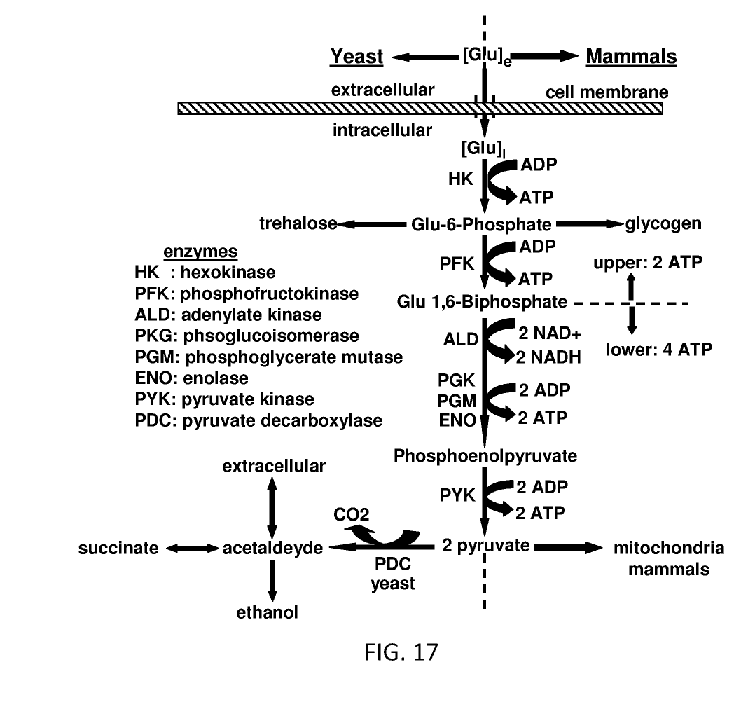

The glucose sensor is indirectly measuring the flux of glucose into the cells and that the flux of glucose across the yeast cell membrane is oscillating, therefore the glucose oxidase sensor also sees an oscillating glucose (Gₑ) concentration. Glucose oscillations from a hybrid yeast cell/glucose sensor, such as those in FIGS. 15A and 15B, have not been previously reported. Previously, glycolytic oscillations in yeast cells were observed as fluctuations in NADH fluorescence. Oscillations in NADH and other cellular metabolites usually occur at periods less than 10 minutes. Before oscillations occur, it has been postulated that an intracellular pool of metabolites must build up to a point where glycolytic oscillations become visible, in this experiment it took about 600 min for this process to occur. As shown in FIG. 17, this is in line with assumptions regarding the nature of glycolytic oscillations in yeast being centered on phosphofructokinase (PFK). The imbalance between inhibitors, activators and reaction kinetics on PFK create characteristic oscillations in the flux of glucose into yeast cells and by-products of glycolysis such as ATP and NADH. The allosteric enzyme PFK is also the oscillophore present in mammalian cells, including fibroblasts.

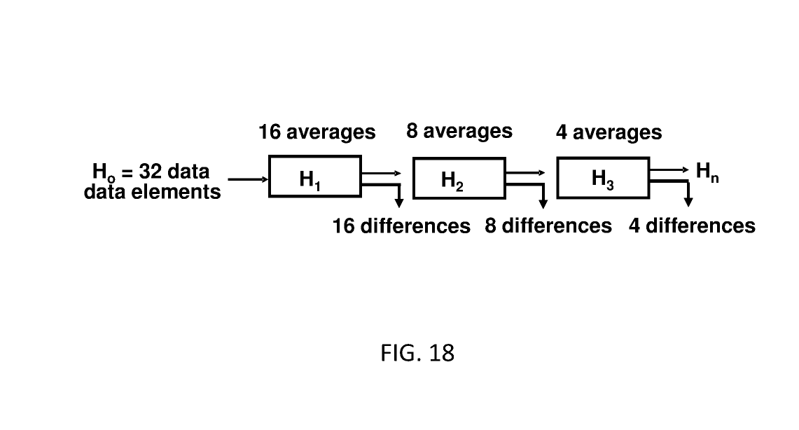

To characterize the oscillations observed in FIG. 15, a simple wavelet technique was utilized based on the Haar Transform. As shown in FIG. 18, the basis of the Haar wavelet is a “lifting scheme” consisting of two parts, one is an average or low frequency component and the second is a difference or high frequency component. The high frequency component contains most of the noise. At each stage of the averaging process, the initial signal can be re-constructed using the averages and differences in the reverse order. In our analyses, we were interested in the low frequency component of the data, i.e., averaged glucose.

The data sampling rate was 5 seconds (0.2 Hz). To construct a wavelet using the Haar Transform, discrete, non-overlapping, consecutive two point averages of the sensor output signal are taken on a set of an even number, Hₒ (N), of data points (2ⁿ). The first set (H₁) of averages results in N/2 data points with the time (period) between data points increasing by a factor of 2. For H₁, the period is 5 sec*2 which is 10 seconds. Each subsequent Hₙ has associated with it a unique average frequency. The wavelet algorithm is recursive and the averaged data from the first step (H₁) becomes the input for the next step (H₂), with halving of the number of data points and doubling of the period (20 seconds), until there are no more data points to average. At each subsequent Hₙ step, the first or second order differencing of the averaged glucose data is used to extract the oscillation data.

By using this simple scheme, the data is filtered (by averaging) as it moves from one Hₙ step to the next. For yeast cell experiments, the glucose concentration was held constant such that the point-to-point, first order differences (dG₁) in the signal were used to extract periodic data. When the glucose is held constant, the 1st order difference also serves to detrend the data. Using a peak finder algorithm, the time points corresponding to the maxima in the series of oscillating data points (dG₁) were determined. The data was sorted according to the maxima and the time difference between each successive maximum dG₁ calculated. These time points were averaged and the standard deviation calculated. FIGS. 15A and 15B are an expanded view of FIG. 14 and show a series of yeast glucose oscillations between 650 and 825 minutes obtained from the data in FIG. 14. The peak detection algorithm was used on the H₄, dG₁ data. The average period was 5.8 minutes±1.5 minutes (FIG. 15B). The calibrated average maximum from the Haar H₄ level of the positive glucose oscillations was about +50 mg/dL and the average minimum of the negative glucose oscillations was about −50 mg/dL. Since the average bulk concentration of glucose was about 220 mg/dl, the average amplitude of the glucose oscillations were about ±23% of the bulk glucose concentration.

### Example 2

A controlled clinical study was conducted to validate in vivo, the in vitro model developed using yeast cells. The clinical study was approved by the Internal Review Board of a Clinical Research Unit of a hospital and was designed to obtain subcutaneous, continuous glucose sensor data from normal human subjects (N=5), and subjects with type 1 (N=5) and type 2 (N=5) diabetes for the purposes of measuring in vivo glucose oscillations within the dermis. Study subjects participated in a supervised 12-hour in-clinic study. Each subject was fitted with two continuous glucose monitors (CGMs), one on each side of the abdominal region.

The time series of glucose data generated from the continuous glucose sensors was analyzed to: (1) confirm the presence of glucose oscillations within subcutaneous interstitial fluid; (2) compare the characteristics of subcutaneous glycemic oscillations in humans to those of the in vitro yeast model system and (3) to determine the amplitude and frequency of the in vivo oscillations. The sensor output currents from the intradermal CGM sensor were calibrated versus simultaneous venous blood glucose measurements made independently using a Yellow Springs Instruments Model 2300 Glucose Analyzer as a laboratory reference method.

In Vivo ISF Glucose Oscillations: The process for measuring in vivo, subcutaneous glucose oscillations was the same used to measure glucose oscillations in yeast; however, a distinguishing feature of the in vivo subcutaneous measurements was cells were not directly immobilized or trapped on the sensor surface. The presence of the glucose sensor in the dermal space allows cells such as fibroblasts to come into proximal contact with the sensor surface and thereby allow for the measurement of the metabolic flux of glucose at the cellular interface to be determined.

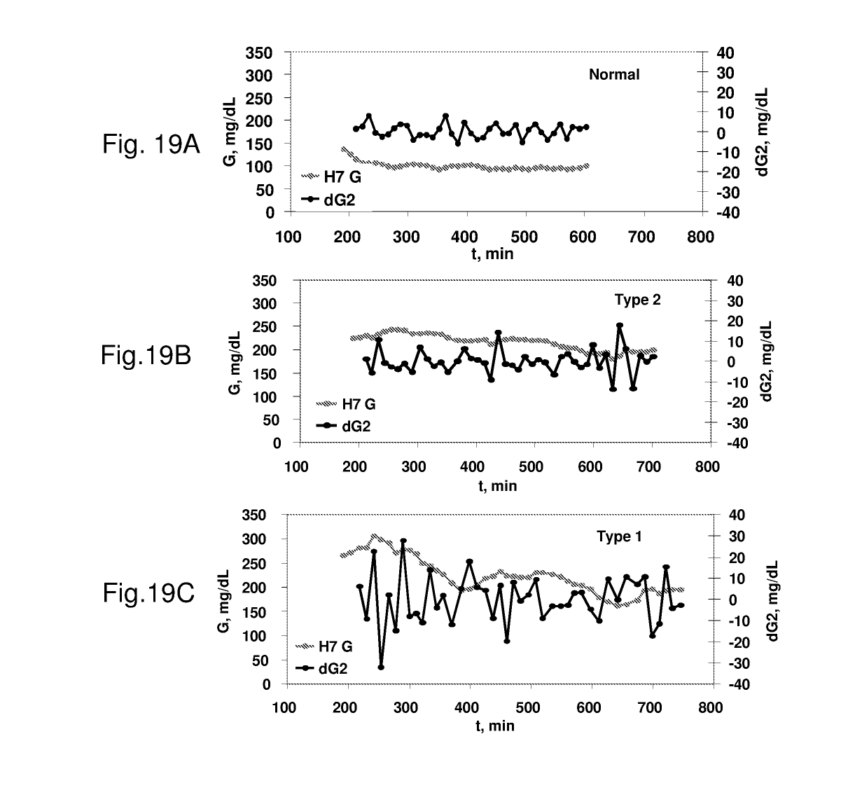

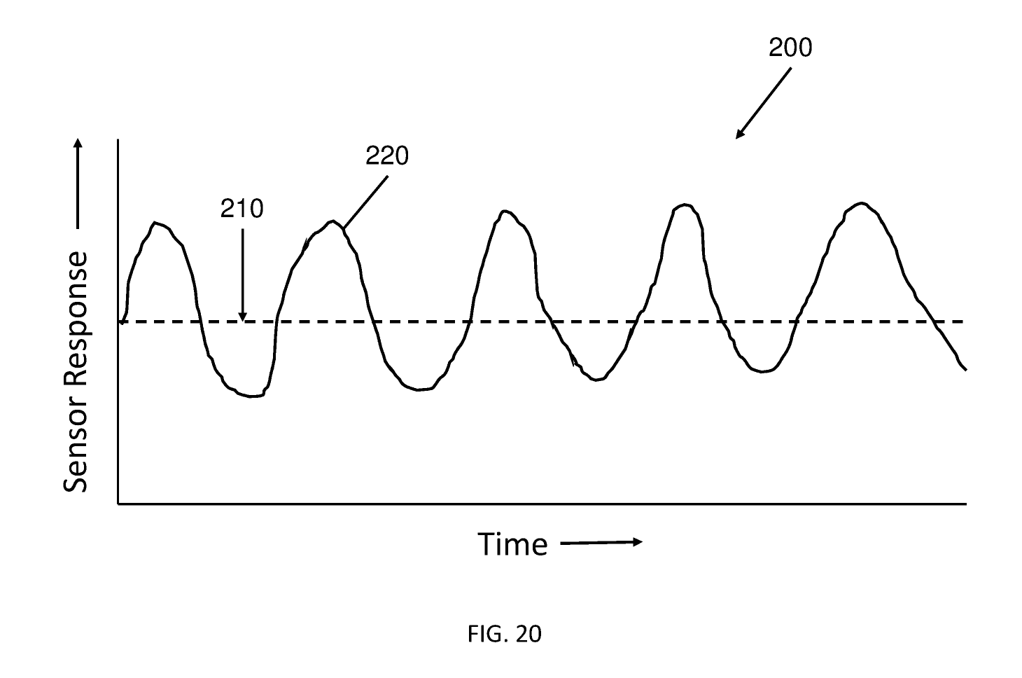

The raw data from the subcutaneous sensor responses was initially smoothed using an exponential moving average which minimized the variance between measurements. The same Haar Wavelet method, as described in Example 1, was used to extract glucose oscillation data. FIG. 19 shows three graphs FIG. 19A, FIG. 19B and FIG. 19C of subcutaneous glucose responses from a normal individual and individuals with type 2 and type 1 diabetes, respectively. FIGS. 19A, B & C serve to demonstrate some general observations. The left Y-axis of the graphs in FIG. 19 is the filtered, calibrated, bulk glucose concentration of the subcutaneous sensor (gray trace), the right Y-axis is the second order difference dG₂ (black trace) in glucose concentration and the X-axis in all graphs is the time in minutes.

In contrast to the yeast cell experiments, the subcutaneous glucose concentrations were not at a steady state and therefore a second order difference (dG₂) was used to detrend the data. The data in FIGS. 19 A-C was obtained from the H₇ level of the Haar transform. Generally, the period increases as Hₙ increases and the average period of dG₂ oscillations at H₇ was 33.4±3.8 min. At lower Hₙ levels, the period decreases and the noise increases. In this analysis, an H₅ or greater level was used. On average, the H₇ dG₂ glucose oscillations increased in amplitude going from normal to type 2 to type 1 diabetes (3.6 mg/dL, 4.5 mg/dL, and 8.2 mg/dL, respectively). The average glucose concentration corresponding to the dG₂ peak oscillations also increased in the same manner (102 mg/dL, 172 mg/dL, 208 mg/dL; respectively, from normal to type 2 to type 1 diabetes). The trends held for lower Hₙ levels (e.g. H₅ & H₆). The oscillation data obtained in this experiment showed there were differences in amplitude of the glucose oscillations in normal versus type 1 and type 2 diabetes. The data are indicative of variations in the amplitude of subcutaneous glucose oscillations, over periods of less than one hour, and can be used to determine the glycemic state of an individual, screen for early signs of diabetes or provide data to better control an insulin pump.

The periods measured in this experiment fall within the region of lower frequency oscillations (<1 hour) which are more related to exogenous inputs such as meals and insulin doses which are more meaningful for controlling glucose levels. In addition, since there is little or no beta cell activity in type 1 subjects, it was previously believed that subcutaneous glucose measurements should not exhibit periodic signals indicative of pulsatile insulin secretion. In contrast to the prior art, this invention shows that cells in close proximity to the implanted glucose sensor should exhibit oscillatory glucose metabolism because the enzymatic pathway in mammalian and yeast cells share a common path linked to the enzyme phosphofructokinase which causes glycolysis to oscillate. Furthermore, the data from this experiment shows that cellular glucose oscillations within the subcutaneous tissue are present regardless of whether the subject has type 1 diabetes.

In addition, Experiment 2 shows that it is possible to accurately measure relatively small variations in glucose concentration independent of background noise and thereby provide a useful measure for evaluating type 1 diabetes, type 2 diabetes, pre-diabetic conditions, and metabolic syndrome.

1-209. (canceled)

210. A method of obtaining a metabolic state from a pattern of metabolite oscillations in a subject, comprising:
inserting a metabolite specific biosensor within the skin of the subject;
applying energy to the biosensor;
recording and storing output metabolite response data from the biosensor for a period of time;
filtering the output response data using time series analysis to provide filtered response data;
calibrating the filtered response data versus metabolite concentration;
obtaining periodic sensor response data corresponding to concentrations of metabolite in the subject over the period of time;
converting the periodic sensor response data to a time series pattern consisting of changes in sensor response from point to point, and
comparing the time series pattern to characteristic patterns of different metabolite oscillations corresponding to metabolic states to determine the metabolic state of the subject.

211. The method of claim 210, wherein the metabolite is selected from the group consisting of glucose, lactate, pyruvate, adenosine triphosphate, adenosine diphosphate, NAD⁺, NADH and phosphofructokinase.

212. The method of claim 210, wherein the subject is a human.

213. The method of claim 210, wherein the metabolite is glucose.

214. The method of claim 210, wherein the metabolic state is a state of glycemia.

215. The method of claim 214, wherein the state of glycemia is selected from the group consisting of type 1 diabetes, type 2 diabetes, impaired glucose tolerance, pre-diabetic, metabolic syndrome, normal and combinations thereof.

216. The method of claim 210, wherein the characteristic patterns of different metabolic states comprise amplitude and frequency data.

217. The method of claim 210, wherein the biosensor output response data is electrical, optical, electromagnetic, or a combination thereof.

218. The method of claim 210, wherein the period of time can be selected from seconds, minutes, hours, days, weeks, months, or years.

219. The method of claim 210, wherein the time series analysis is selected from the group consisting of wavelet analysis, Fourier transform, spectral density analysis and combinations thereof.

220. A system for measuring a metabolic state from a pattern of metabolic oscillations in a subject and characteristic patterns of metabolic states, comprising:
a biosensor inserted within the skin of the subject;
a computer readable media for recording and storing output response data from the biosensor for a period of time;
filtering means for carrying out time series analysis of the output response data to provide filtered response data;
means for extracting a pattern of metabolic oscillations from periodic sensor response data that has been calibrated versus metabolite concentration and corresponds to concentrations of a metabolite in the subject over the period of time, and
means for converting the periodic sensor response data to a time series pattern consisting of changes in sensor response from point to point and comparing the time series pattern to characteristic patterns of different metabolic states to obtain the metabolic state of the subject.

221. The system of claim 220, wherein the metabolite is selected from the group consisting of glucose, lactate, pyruvate, adenosine triphosphate, adenosine diphosphate, NAD⁺, NADH and phosphofructokinase.

222. The system of claim 221, wherein the metabolite is glucose.

223. The system of claim 220, wherein the biosensor output response data is electrical, optical, electromagnetic or a combination thereof.

224. The system of claim 220, wherein the time series analysis is obtained using wavelet, Fourier transform or spectral density analysis.

225. A device for obtaining a metabolic state from a pattern of metabolic oscillations in a subject, comprising:
a metabolite specific biosensor configured for mounting within the skin of the subject;
a computer readable media for recording and storing output response data from the biosensor for a period of time;
filtering means for carrying out time series analysis of the output response data to provide filtered response data;
means for extracting a pattern of metabolic oscillations from periodic sensor response data that has been calibrated versus metabolite concentration and corresponding to concentrations of a metabolite in the subject over the period of time, and
means for converting the periodic sensor response data to a time series pattern consisting of changes in sensor response from point to point and
comparing the time series pattern to characteristic patterns of different metabolic states to obtain the metabolic state of the subject.

226. The device of claim 225, wherein the metabolite specific biosensor is specific for a metabolite selected from the group consisting of glucose, lactate, pyruvate, adenosine triphosphate, adenosine diphosphate, NAD⁺, and NADH phosphofructokinase.

227. The device of claim 225, wherein the wherein the metabolite specific biosensor is coated with immobilized cells.

228. The device of claim 227, wherein the immobilized cells are in an in vitro environment.

229. The device of claim 227, wherein the immobilized cells are selected from eukaryotic cells or prokaryotic cells.

230. The device of claim 228, wherein the eukaryotic cells are selected from yeast cells, cancer cells, stem cells or T-cells.
